SEQUENCE LISTING (11 sequences)

SEQ ID NO: 1 - protein, 13 aa, Artificial Sequence
  Description of Artificial Sequence Synthetic peptide
AHIVMVDAYKPTK

SEQ ID NO: 2 - protein, 14 aa, Artificial Sequence
  Description of Artificial Sequence Synthetic peptide
VPTIVMVDAYKRYK

SEQ ID NO: 3 - protein, 115 aa, Artificial Sequence
  Description of Artificial Sequence Synthetic polypeptide
AMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 4 - protein, 115 aa, Artificial Sequence
  Description of Artificial Sequence Synthetic polypeptide
AMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 5 - protein, 13 aa, Artificial Sequence
  Description of Artificial Sequence Synthetic peptide
SRLEEELRRRLTE

SEQ ID NO: 6 - protein, 16 aa, Artificial Sequence
  Description of Artificial Sequence Synthetic peptide
RDHMVLHEYVNAAGIT

SEQ ID NO: 7 - DNA, 200 bp, Artificial Sequence
  Description of Artificial Sequence Synthetic polynucleotide
tttccgggacccctgccccgcgggcagcgctgccaacctgccggccatgcgtgaccacatggtccttcatgagtatgtaaatgctgctgggattacaggttctgtgcctactatcgtgatggtggacgcctacaagcgttacaagggatccgagaccccgtcccagcggcgcgccacccgcagcggggcgcaggccagct

SEQ ID NO: 8 - DNA, 200 bp, Artificial Sequence
  Description of Artificial Sequence Synthetic polynucleotide
cctttccgggacccctgccccgcgggcagcgctgccaacctgccggccatgcctagccgcctggaggaagaactccgccgacgattgactgagccaggttctgtgcctactatcgtgatggtggacgcctacaagcgttacaagggatccgagaccccgtcccagcggcgcgccacccgcagcggggcgcaggccagctc

SEQ ID NO: 9 - DNA, 200 bp, Artificial Sequence
  Description of Artificial Sequence Synthetic polynucleotide
gtccttcgacccgagccccgcgccctttccgggacccctgccccgcgggcagcgctgccaacctgccggccatgcgtgaccacatggtccttcatgagtatgtaaatgctgctgggattacaggatccgagaccccgtcccagcggcgcgccacccgcagcggggcgcaggccagctccactccgctgtcgcccacccgc

SEQ ID NO: 10 - DNA, 200 bp, Artificial Sequence
  Description of Artificial Sequence Synthetic polynucleotide
tgtccttcgacccgagccccgcgccctttccgggacccctgccccgcgggcagcgctgccaacctgccggccatgcctagccgcctggaggaagaactccgccgacgattgactgagccaggatccgagaccccgtcccagcggcgcgccacccgcagcggggcgcaggccagctccactccgctgtcgcccacccgcat

SEQ ID NO: 11 - DNA, 200 bp, Artificial Sequence
  Description of Artificial Sequence Synthetic polynucleotide
tctgtccttcgacccgagccccgcgccctttccgggacccctgccccgcgggcagcgctgccaacctgccggccatggtgcctactatcgtgatggtggacgcctacaagcgttacaagggatccgagaccccgtcccagcggcgcgccacccgcagcggggcgcaggccagctccactccgctgtcgcccacccgcatc

(19) United States
(12) Patent Application Publication
(10) Pub. No.: US 2024/0003893 A1
(43) Pub. Date: Jan. 4, 2024

(54) RECONSTITUTION OF A SPLIT-HALOTAG VIA ORTHOGONAL TAG-BINDING DOMAINS

(71) Applicants: CZ Biohub SF, LLC, San Francisco, CA (US); The Regents of the University of California, Oakland, CA (US)

(72) Inventors: Bo Huang, San Francisco, CA (US); Suraj Makhija, San Francisco, CA (US); David Brown, Oakland, CA (US)

(21) Appl. No.: 18/253,718

(22) PCT Filed: Nov. 29, 2021
(86) PCT No.: PCT/US2021/061001

§ 371 Date: May 19, 2023

Related U.S. Application Data
(60) Provisional application No. 63/119,160, filed on Nov. 30, 2020.

(51) Int. Cl. G01N 33/58; G01N 33/68

(52) U.S. Cl. CPC G01N 33/582; G01N 33/68

(57) ABSTRACT

We have developed the tag-assisted split enzyme complementation (TASEC) approach, which uses two orthogonal small peptide tags and their cognate binders to conditionally drive complementation of a split enzyme upon labeled protein expression. Using this approach, we have engineered and optimized the tag-assisted split HaloTag complementation system (TA-splitHalo) and demonstrated its versatile applications in improving the efficiency of knock-in cell enrichment, detection of protein-protein interaction, and isolation of biallelic gene edited cells through multiplexing.

### CROSS-REFERENCE TO RELATED PATENT APPLICATIONS

The present application claims benefit of priority to U.S. Provisional Patent Application No. 63/119,160, filed Nov. 30, 2020, which is incorporated by reference for all purposes.

### STATEMENT REGARDING FEDERALLY FUNDED RESEARCH

This invention was made with government support under grants R21 GM129652, R01 GM131641, and R01 CA231300 awarded by The National Institutes of Health. The government has certain rights in the invention.

### BACKGROUND OF THE INVENTION

CRISPR/Cas9-mediated genome engineering techniques have revolutionized the study of endogenous biology. With these techniques, one powerful application is to label proteins by genomic knock-in so that the abundance, dynamics, and interactions of endogenous proteins can be examined while avoiding artifacts of overexpression. For this purpose, one approach is to use fluorescent protein (FP) fusions, enabling the use of fluorescence activated cell sorting (FACS) to directly isolate and enrich for knocked-in (KI) cells. However, the large size of FPs leads to potential perturbation of the tagged protein's localization and function and more importantly, impacts the efficiency and scalability of the knock-in approach.

In contrast, short peptide tags can be used to overcome these limitations, but they are not inherently fluorescent and are not compatible with live cell FACS unless the tag is extracellularly localized and therefore compatible with antibody staining. An alternative option is split fluorescent protein, or FP₁₁ tags, which were developed based on the self-complementing split GFP_{1-10/11} [Cabantous, S., Terwilliger, T. C. & Waldo, G. S., Nature Biotechnology 23, 102-107 (2005); Kamiyama, D. et al., Nature Communications 7, 11046 (2016)] and the split of mNeonGreen and sfCherry [Kamiyama, D. et al., Nature Communications 7, 11046 (2016); Feng, S. et al., Nature Communications 8, 370 (2017); Feng, S. et al., Communications Biology 2, 1-12 (2019)]. These tags are 16 a.a. peptides derived from the 11^{th} β strand of FPs. Once expressed, the corresponding FP₁₋₁₀ fragment will bind FP₁₁ tags to form a functional FP. Owing to their combined small size and fluorescence, FP₁₁ tags have greatly facilitated the generation and analysis of mammalian cell libraries containing endogenously tagged proteins [Leonetti, M. D. et al., PNAS 113, E3501-E3508 (2016)].

Still, FP₁₁ tags have intrinsic limitations in fluorophore brightness and photostability, making it challenging to detect and track low expression targets. Moreover, it is highly desirable to expand this tagging approach to other split protein complementation systems, such as split luciferase for bioluminescence detection [Paulmurugan, R. & Gambhir, S. S., Anal Chem 75, 1584-1589 (2003)], split protease for synthetic circuits [Gao, X. J. et al., Science 361, 1252-1258 (2018)], and split enzymatic tags, particularly split HaloTag [Ishikawa, H. et al., Protein Engineering Design and Selection 25, 813-820 (2012)], that enable labeling of the target protein with organic fluorophores that are bright, photostable and available in many different colors. This also would enable reporter outputs beyond fluorescence. Unfortunately, none of these split proteins are self-complementing, meaning that they require additional protein-recruitment strategies to induce the complementation of the split fragments. In addition, the roughly central position of their split points means that neither fragment is small enough to serve as a short peptide tag. Therefore, they cannot be directly adapted to endogenous protein tagging like the split FP_{1-10/11} systems.

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art.

A “target protein” refers to any protein that can be expressed in, or otherwise introduced into, a cell of interest and for which measurement of expression, localization and/or interaction is desired. The target protein amino acid sequence will be linked to at least one, and possibly to different peptide tags as described herein to form a fusion target protein.

A “fusion protein” refers to a single polypeptide that comprises two heterologous polypeptide sequences that are linked together via a peptide bond and optionally a peptide linker. Each heterologous polypeptide can be for example at least 5, 10, 20 or more amino acids long. In some embodiments, the fusion protein can be a target protein fused to one or more peptide tags. In some embodiments, the fusion protein can be an affinity agent that specifically binds to a peptide tag, wherein the affinity agent is fused with a portion of a split reporter.

A polypeptide sequence is “heterologous” to a second polypeptide sequence if it originates from a foreign species, or, if from the same species, is modified from its original form, or if it is artificially designed or evolved. For example, when a first polypeptide is linked to a second polypeptide, that is heterologous, it means that the first polypeptide is derived from one species whereas the second polypeptide sequence is derived another, different species; or, if both are derived from the same species, the first polypeptide sequence is not naturally associated with the second polypeptide sequence (e.g., is a genetically engineered to be fused together).

A “split reporter” protein refers to a protein which generates a signal, e.g., via substrate binding activity (see, e.g., To et al., Protein Science 2016, 25, 748-753) and/or enzyme activity (see, e.g., Wehr et al., Nature Methods, 2006, 3, 985-993) of the protein, when two portions of the protein are brought into proximity. The split reporter can be generated for example by splitting a single protein having enzymatic activity that results in a signal into two portions that when combined together in solution and brought within proximity to each other generate detectable signal, which is optionally at least most (at least 50%, 70%, 90%) of the signal that the intact reporter generates. Ishikawa, et al., Protein Engineering, Design and Selection, Volume 25, Issue 12, Dec. 2012, Pages 813-820, for example describes methods for identifying active portions of a reporter protein and methods for testing and confirming portions retain the activity of the intact reporter when the portions are in proximity to each other. The portions of the split reporter can, but need not necessarily, include all of the amino acids of the intact reporter protein.

“Proximity” in the context of this disclosure, means that the two split reporter portions are brought close enough to generate signal that is distinguishable from background signal when the two split reporter portions are in solution together without an affinity agent or peptide tag to bring them together.

“Detection protein” is sometimes used herein to refer to a fusion of an affinity agent and a portion of a split reporter protein. To generate a signal, two detection proteins, comprising different portions of the split reporter, and different affinity agents than bind respective peptide tags in proximity, are brought together by their binding to the respective peptide tags, allowing for the split reporter portions to form an active enzyme complex, which can be detected by its activity.

The use of “first,” “second,” “third,” etc. in this disclosure is simply for antecedent basis to distinguish other molecules of the same type. For example a “first protein” and a “second protein” means there are two distinguishable proteins. Order is not intended by this usage.

The words “protein”, “peptide”, and “polypeptide” are used interchangeably to denote an amino acid polymer. The terms do not specify a certain length, though peptides are generally shorter than proteins or polypeptides.

A “peptide tag” as used herein refers to a peptide sequence that a corresponding affinity agent has affinity for. The affinity agent will specifically bind to its corresponding peptide tag. Two different peptide tags used herein will be orthogonal, meaning that different affinity agents bind to different peptide tags but they do not significantly cross-react, i.e., the ability of an affinity agent to bind a target peptide tag is at least 10, 20, 50, or 100 fold greater than for a second peptide tag in the same detection system.

The phrase “specifically (or selectively) binds” to a peptide tag refers to a binding reaction whereby the affinity agent binds to the peptide tag of interest. In the context of this disclosure, the affinity agent binds to peptide tag in question with a KD that is at least 100-fold greater than its affinity for other peptide tags in the system or other proteins in the cell in question.

An “affinity agent” refers to a protein sequence that has specific affinity (specifically binds) to a peptide tag sequence as used herein. An affinity agent can be any protein known or selected to have specific affinity for its target peptide tag. Examples of affinity agents include but are not limited to SpyCatcher, SpyCatcher002, NbALFA, GFP1-10, or an antibody (which may be a single-chain scfv antibody or a camelid VHH domain).

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize the RNA-mediated nuclease, Cas9 in complex with guide and activating RNA to recognize and cleave foreign nucleic acid.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24;110(39):15644-9; Sampson et al., Nature. 2013 May 9;497(7448):254-7; and Jinek, et al., Science. 2012 Aug. 17;337(6096):816-21.

### BRIEF SUMMARY OF THE INVENTION

The disclosure provides cells comprising a first fusion protein comprising a first peptide tag and a second peptide tag; a second fusion protein comprising a first portion of a split reporter and a first affinity agent that specifically binds to the first peptide tag; and a third fusion protein comprising a second portion of the split reporter and a second affinity agent that specifically binds to the second peptide tag, wherein the first portion of the split reporter and the second portion of the split reporter produce a first signal when in proximity and are inactive when separate.

In some embodiments, the cell expresses the first fusion protein, the second fusion protein and the third fusion protein. In some embodiments, the cell expresses the first fusion protein and the second fusion protein and the third fusion protein have been introduced as proteins into the cell.

In some embodiments, the peptide tags are each less than 30, 25, 20, 15 or 10 amino acids.

In some embodiments, the split reporter is a HaloTag reporter.

In some embodiments, the first portion of the split reporter is an amino terminal portion of the split reporter and the first affinity agent is linked to the amino terminal side of the first portion and wherein the second portion of the split reporter is a carboxyl terminal portion of the split reporter and the second affinity agent is linked to the carboxyl terminal side of the second portion.

In some embodiments, the signal is fluorescence.

In some embodiments, the first peptide tag and the second peptide tag are adjacent or linked by a linker of fewer than 15 (e.g., fewer than 10, 5, 2) amino acids and are located at the amino terminus of the first fusion protein. In some embodiments, the first peptide tag and the second peptide tag are adjacent or linked by a linker of fewer than 15 (e.g., fewer than 10, 5, 2) amino acids and are located at the carboxyl terminus of the first fusion protein.

In some embodiments, the first peptide tag and the second peptide tag are different and selected from the group consisting of SpyTag, SpyTag002, ALFA-tag, and GFP11 and the corresponding affinity agent is SpyCatcher if the peptide tag is SpyTag, SpyCatcher002 if the peptide tag is SpyTag002, NbALFA if the peptide tag is ALFA-tag, and GFP1-10 if the peptide tag is GFP11. In some embodiments, the first tag is GFP11 and the first affinity agent is GFP1-10, and the second tag is SpyTag and the second affinity agent is SpyCatcher or the second tag is SpyTag002 and the second affinity agent is SpyCatcher002. In some embodiments, the first tag is ALFA-tag and the first affinity agent is NbALFA, and the second tag is SpyTag and the second affinity agent is SpyCatcher or the second tag is SpyTag002 and the second affinity agent is SpyCatcher002. In some embodiments, the first tag is GFP11 and the first affinity agent is GFP1-10, and the second tag is ALFA-tag and the second affinity agent is NbALFA.

In some embodiments, the cell further comprises a fourth fusion protein comprising a GFP11; a fifth fusion protein comprising a GFP1-10; and wherein the first signal of the split reporter is distinguishable from signal from intact GFP.

In some embodiments, the cell further comprises a fourth fusion protein comprising a third peptide tag and a fourth peptide tag; a fifth fusion protein comprising a first portion of a second split reporter and a third affinity agent that specifically binds to the third peptide tag; and a sixth fusion protein comprising a second portion of the second split reporter and a fourth affinity agent that specifically binds to the fourth peptide tag, wherein the first portion of the second split reporter and the second portion of the split reporter produce a signal, distinguishable from the first signal of the split reporter (the signal from the first portion of the split reporter and the second portion of the split reporter), when in proximity and are inactive when separate.

Also provided is a method of selecting cells comprising a heterologous polynucleotide encoding a first fusion protein comprising a target polypeptide and at least two peptide tags. In some embodiments, the method comprises:

modifying the genome of at least some of a plurality of cells with the heterologous polynucleotide encoding the first fusion protein, wherein the first fusion protein comprises a first peptide tag and a second peptide tag and wherein at least some of the plurality of the cells expresses the first fusion protein;
expressing or introducing in the cells: a second fusion protein comprising a first portion of a split reporter and a first affinity agent that specifically binds to the first peptide tag; and a third fusion protein comprising a second portion of the split reporter and a second affinity agent that specifically binds to the second peptide tag, wherein the first portion of the split reporter and the second portion of the split reporter produce a first signal when in proximity and are inactive when separate,
separating a first group of cells in which the split reporter produces the signal from the plurality of cells, thereby selecting cells comprising the heterologous polynucleotide encoding the first fusion protein.

In some embodiments, the method comprises obtaining the plurality of cells from an individual; and after the separating, introducing the first group of cells, or cells expanded therefrom, to the individual.

In some embodiments, the method further comprises modifying the genome of at least some of the plurality of cells with a second heterologous polynucleotide encoding a fusion polypeptide of the target polypeptide and GFP11, wherein at least some of the plurality of the cells expresses the fusion polypeptide; the expressing or introducing comprises expressing or introducing GFP1-10 into the cells; and wherein the first signal is distinguishable from signal from intact GFP, thereby allowing for detection of bi-allelic expression of the target polypeptide.

In some embodiments, the peptide tags are each less than 30, 25, 20, 15 or 10 amino acids.

In some embodiments, the split reporter is a HaloTag reporter.

In some embodiments, the first portion of the split reporter is an amino terminal portion of the split reporter and the first affinity agent is linked to the amino terminal side of the first portion and wherein the second portion of the split reporter is a carboxyl terminal portion of the split reporter and the second affinity agent is linked to the carboxyl terminal side of the second portion.

In some embodiments, the signal is fluorescence.

In some embodiments, the first peptide tag and the second peptide tag are adjacent or linked by a linker of fewer than 15 (e.g., fewer than 10, 5, 2) amino acids and are located at the amino terminus of the first fusion protein. In some embodiments, the first peptide tag and the second peptide tag are adjacent or linked by a linker of fewer than 15 (e.g., fewer than 10, 5, 2) amino acids and are located at the carboxyl terminus of the first fusion protein.

In some embodiments, the first peptide tag and the second peptide tag are different and selected from the group consisting of SpyTag, SpyTag002, ALFA-tag, and GFP11 and the corresponding affinity agent is SpyCatcher if the peptide tag is SpyTag, SpyCatcher002 if the peptide tag is SpyTag002, NbALFA if the peptide tag is ALFA-tag, and GFP1-10 if the peptide tag is GFP11. In some embodiments, the first tag is GFP11 and the first affinity agent is GFP1-10, and the second tag is SpyTag and the second affinity agent is SpyCatcher or the second tag is SpyTag002 and the second affinity agent is SpyCatcher002. In some embodiments, the first tag is ALFA-tag and the first affinity agent is NbALFA, and the second tag is SpyTag and the second affinity agent is SpyCatcher or the second tag is SpyTag002 and the second affinity agent is SpyCatcher002. In some embodiments, the first tag is GFP11 and the first affinity agent is GFP1-10, and the second tag is ALFA-tag and the second affinity agent is NbALFA.

Also provided is a cell comprising:
a first fusion protein comprising a first peptide tag;
a second fusion protein comprising a second peptide tag;
a third fusion protein comprising a first portion of a split reporter and a first affinity agent that specifically binds to the first peptide tag; and
a fourth fusion protein comprising a second portion of the split reporter and a second affinity agent that specifically binds to the second peptide tag,
wherein the first portion of the split reporter and the second portion of the split reporter produce a signal when in proximity and are inactive when separate.

In some embodiments, the cell expresses the first fusion protein, the second fusion protein, the third fusion protein and the fourth fusion protein. In some embodiments, the cell expresses the first fusion protein and the second fusion protein, and the third fusion protein and the fourth fusion protein have been introduced into the cell.

In some embodiments, the peptide tags are each less than 30, 25, 20, 15 or 10 amino acids.

In some embodiments, the split reporter is a HaloTag reporter.

In some embodiments, the first portion of the split reporter is an amino terminal portion of the split reporter and the first affinity agent is linked to the amino terminal side of the first portion and wherein the second portion of the split reporter is a carboxyl terminal portion of the split reporter and the second affinity agent is linked to the carboxyl terminal side of the second portion.

In some embodiments, the signal is fluorescence.

In some embodiments, the first peptide tag and the second peptide tag are located at the amino terminus of the first fusion protein and second fusion protein, respectively. In some embodiments, the first peptide tag and the second peptide tag are located at the carboxyl terminus of the first fusion protein and the second fusion protein, respectively.

In some embodiments, the first peptide tag and the second peptide tag are different and selected from the group consisting of SpyTag, SpyTag002, ALFA-tag, and GFP11 and the corresponding affinity agent is SpyCatcher if the peptide tag is SpyTag, SpyCatcher002 if the peptide tag is SpyTag002, NbALFA if the peptide tag is ALFA-tag, and GFP1-10 if the peptide tag is GFP11. In some embodiments, the first tag is GFP11 and the first affinity agent is GFP1-10, and the second tag is SpyTag and the second affinity agent is SpyCatcher or the second tag is SpyTag002 and the second affinity agent is SpyCatcher002. In some embodiments, the first tag is ALFA-tag and the first affinity agent is NbALFA, and the second tag is SpyTag and the second affinity agent is SpyCatcher or the second tag is SpyTag002 and the second affinity agent is SpyCatcher002. In some embodiments, first tag is GFP11 and the first affinity agent is GFP1-10, and the second tag is ALFA-tag and the second affinity agent is NbALFA.

Also provided is a method of measuring protein-protein interaction, the method comprising
providing the cell comprising:
a first fusion protein comprising a first peptide tag;
a second fusion protein comprising a second peptide tag;
a third fusion protein comprising a first portion of a split reporter and a first affinity agent that specifically binds to the first peptide tag; and
a fourth fusion protein comprising a second portion of the split reporter and a second affinity agent that specifically binds to the second peptide tag,
wherein the first portion of the split reporter and the second portion of the split reporter produce a signal when in proximity and are inactive when separate; and
measuring the presence or amount of the signal from the cell.

Also provided is a cell expressing or comprising:
a first fusion protein comprising a first portion of a split reporter and a first affinity agent that specifically binds to a first peptide tag; and
a second fusion protein comprising a second portion of the split reporter and a second affinity agent that specifically binds to a second peptide tag,
wherein the first portion of the split reporter and the second portion of the split reporter produces a signal when in proximity and are inactive when separate.

In some embodiments, the split reporter is a HaloTag reporter.

In some embodiments, the first portion of the split reporter is an amino terminal portion of the split reporter and the first affinity agent is linked to the amino terminal side of the first portion and wherein the second portion of the split reporter is a carboxyl terminal portion of the split reporter and the second affinity agent is linked to the carboxyl terminal side of the second portion.

In some embodiments, the signal is fluorescent.

In some embodiments, the first affinity agent and the second affinity agent are different and selected from the group consisting of SpyCatcher, SpyCatcher002, NbALFA, and GFP1-10. In some embodiments, the first affinity agent is GFP1-10, and the second affinity agent is SpyCatcher or the second affinity agent is SpyCatcher002. In some embodiments, the first affinity agent is NbALFA, and the second affinity agent is SpyCatcher or the second affinity agent is SpyCatcher002. In some embodiments, the first affinity agent is GFP1-10, and the second affinity agent is NbALFA.

### BRIEF DESCRIPTION OF THE DRAWINGS

FIG. 1A-B: TA-splitHalo Overview and Applications. FIGS. 1A-B. (FIG. 1A) A schematic of the Tag-Assisted Split Enzyme Complementation (TASEC) concept as applied to TA-splitHalo. (1) Two orthogonal peptide tags are knocked-in on a target protein (2) Cognate binders fused to the two unfolded splitHalo fragments are recruited to the tags (3) Confinement of the splitHalo fragments drives refolding of a functional HaloTag molecule. (FIG. 1B) The TA-splitHalo strategy can be applied to tag proteins by knocking-in both tags on the same target protein (left), protein interactions by knocking-in individual tags on interacting proteins (center), or tagging multiple alleles by relegating different TA-splitHalo approaches to different alleles in the same cell (right).

FIG. 2: TA-splitHalo Architecture Scanning. (FIG. 2A) Schematic of GFP/Spy co-transfection architecture scan. Cells were transfected with a plasmid that expresses each GFP/Spy TA-splitHalo architecture and an mCherry bait expression vector tagged with SpyTag (SpyT) alone or GFP11-SpyT. (FIG. 2B) Raw flow cytometry depicting GFP/Spy TA-splitHalo signal (y-axis) vs. MCherry tag reporter expression (x-axis). Each plot is a random sampling of 10k singlet-gated events for each architecture with a SpyT-mCherry bait (grey) or GFP11-SpyT-mCherry (green). (FIG. 2C) Mean hit-rate of each GFP/Spy splitHalo architecture in samples with SpyT-mCherry (grey) or GFP11-SpyT-mCherry (green). (FIG. 2D) Schematic of ALFA/Spy co-transfection architecture scan. Cells were transfected with a plasmid that expresses each ALFA/Spy TA-splitHalo architecture and an mCherry bait expression vector tagged with SpyT alone or ALFA-SpyT. (FIG. 2E) Raw flow cytometry depicting ALFA/Spy TA-splitHalo signal (y-axis) vs. mCherry tag reporter expression (x-axis). Each plot is a random sampling of 10k singlet-gated events for each architecture with a SpyT-mCherry bait (grey) or ALFA-SpyT-mCherry (orange). (FIG. 2F) Mean hit-rate of each ALFA/Spy splitHalo architecture in samples with SpyT-mCherry (grey) or ALFA-SpyT-mCherry (orange). Statistical significance of differences between hit rates for both GFP/Spy and ALFA/Spy systems was determined by Welch's t-test, comparing the mean of biological triplicates (n=3).

FIG. 3A-F: Evaluating Signal to Background of TA-splitHalo Architectures in Single-Copy Cell Lines. (FIG. 3A) Overview of knock-in strategy in single-copy detection cell lines. Short tag knock-ins on the LMNA gene were performed in cell lines pre-engineered to express the requisite detection components for each detection system off a single transcriptional unit at the same genomic locus. (FIG. 3B) Table depicts illustrations of the relevant proteins expressed in knock-in lines. (FIG. 3C) Median signal intensity in the GFP channel in background (grey) and knock-in (green) conditions in each detection cell line. (FIG. 3D) Median signal intensity in the TA-splitHalo channel in background (grey) and knock-in (red) conditions in each detection cell line. Median is derived from flow cytometry data of 10k cells per condition. (FIG. 3E) Signal to background values calculated by taking the ratio of knock-in to background GFP median signal (green) and TA-splitHalo signal (red) for each detection system. Dashed line depicts 1:1 signal to background detection threshold. (FIG. 3F) Confocal images of all LMNA knock-ins in detection cell line. Panels show nuclear BFP integration reporter (top row) LMNA-specific splitGFP signal (center row), and LMNA-specific TA-splitHalo signal (bottom row).

FIG. 4A-I. TA-splitHalo can detect interactions between ALFA-LMNA and SpyTag-LMNA. (FIG. 4A) Overview of knock-in strategy for TA-splitHalo detection of self-associating lamin A/C chains. Knock-In is performed with both ALFA and SpyT donor strands (FIG. 4B) In cells that contain both knock-ins, tags are in proximity upon dimerization of lamin chains.

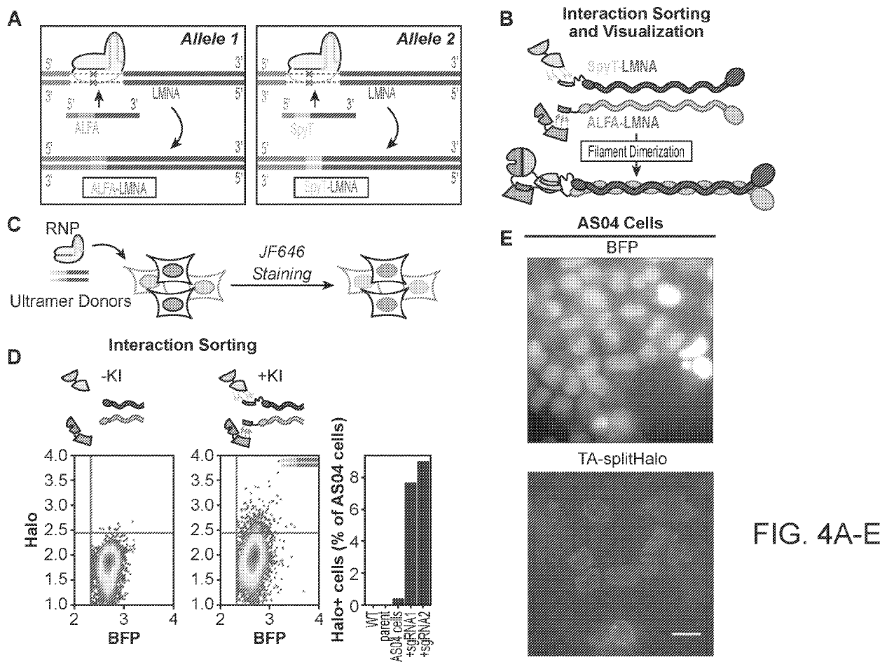

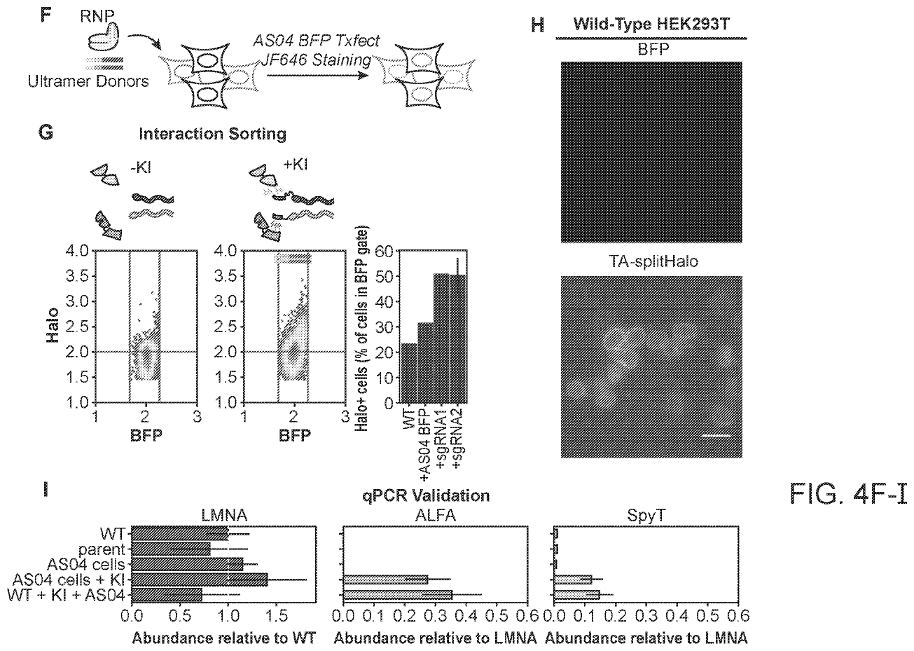

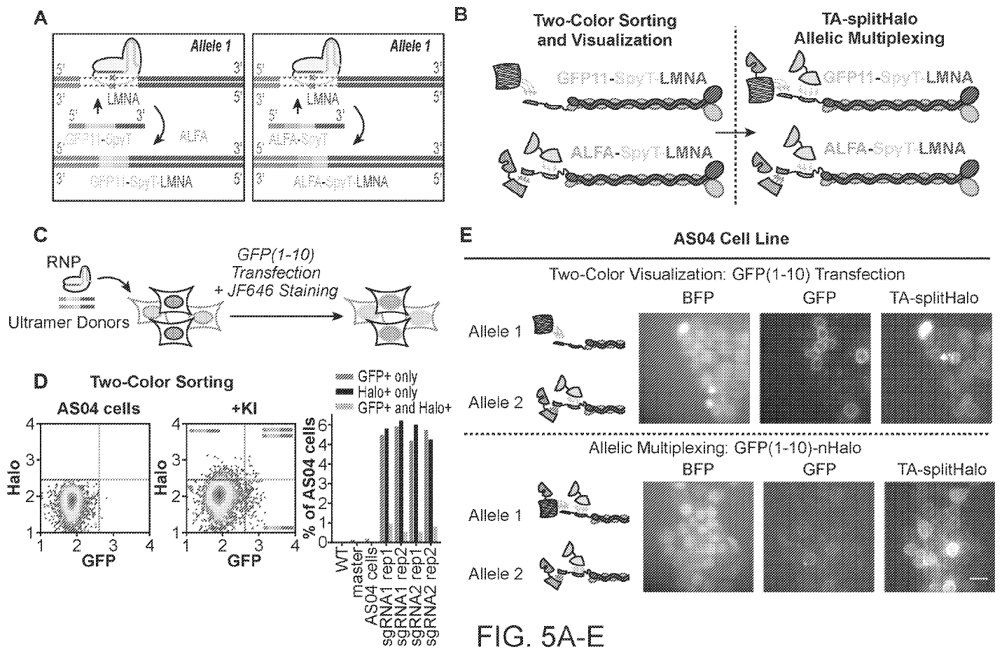

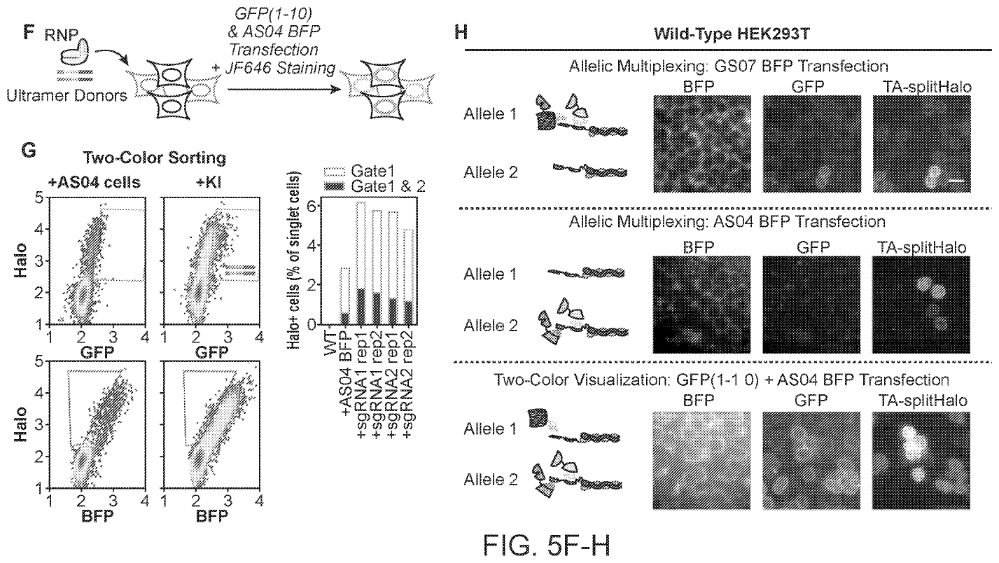

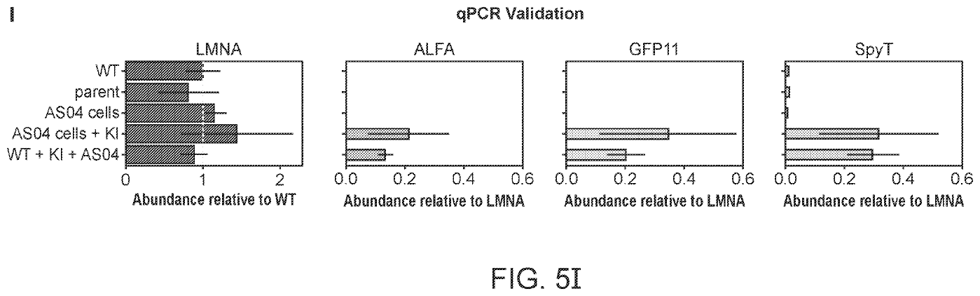

(FIG. 4C) Experimental workflow for ALFA/Spy TA-splitHalo interaction knock-ins in the AS04 cell line. The knock-ins shown in Panel A is performed in the AS04 detection cell line which constitutively expresses NbALFA-nHalo and SpyCatcher-cHalo as well as NLS-TagBFP from a single copy locus. Staining with 10 nM JF646 yields TA-splitHalo nuclear envelope labelling.

(FIG. 4D) Flow cytometry data from knock-ins performed in the AS04 stable cell line as shown in panel A. Events (3000 per panel) are shown on log10 scale, and have been gated to show mRuby−, TagBFP+ single cells as described in the methods section. The left panel is a control showing the unedited AS04 cell line. From this cell population, we set gates to threshold for BFP+ cells (blue line) and TA-splitHalo+ cells (red line). The center panel shows AS04 cells, nucleofected with Cas9 complexed with sgRNA targeting LMNA and an equimolar mixture of single stranded donor DNA introducing either the ALFA peptide or the SpyT peptide. Cells in the top right quadrant of this plot were sorted to enrich for ALFA/SpyT-LMNA cells. The bar graphs shows the percentage of Halo+ cells for WT HEK293Ts (0), the master cell line (0), our AS04 cells with no KI, or AS04 cells after Cas9 targeting of LMNA with both ALFA- and Spy-donors. The distinct bars for the +KI group represent sgRNA1 (left) and sgRNA2 (right) targeting LMNA.

(FIG. 4E). A representative widefield microscopy image of cells sorted in panel D. Imaging at 405 nm shows the single copy NLS-TagBFP constitutively expressed by the AS04 cell line. The 646 nm image reveals complementation of the AS04 splitHalo system in a subset of cells. The 646 nm signal follows the nuclear outline, reflecting the recruitment of nHalo and cHalo to the nuclear lamina by cells expressing both ALFA-LMNA and SpyT-LMNA. The scale bar represents 25 μm.

(FIG. 4F) Experimental workflow for ALFA/Spy TA-splitHalo interaction knock-ins in wild-type HEK293Ts. In order to detect successful lamin knock-ins, we transfected the ASBLU04 TA-splitHalo plasmid and stained with 10 nM JF646. (G) Flow cytometry data from knock-ins performed in the wild-type cell line as shown in panel F. Events (3000 per panel) are shown on log10 scale, and have been gated to show cells with low background expression of TA-splitHalo (blue lines). The left panel is a control showing the wild-type cell line with the ASBLU04 transfection. From this condition, we fit a gate to screen for TA-splitHalo+ cells on the FACS machine (red line). An optimized gate is also shown generated using Python tools (dashed red line). The center panel shows wild-type cells, nucleofected with Cas9 complexed with sgRNA targeting LMNA and an equimolar mixture of single stranded donor DNA introducing either the ALFA peptide or the SpyT peptide. Cells in the top right quadrant of this plot were sorted to enrich for ALFA-/SpyT-LMNA cells. The bar graphs in the right panel shows the percentage of TA-splitHalo+ cells. (H). A representative widefield microscopy image of cells sorted in panel G. Imaging at 405 nm shows the single copy NLS-TagBFP constitutively expressed by the AS04 cell line and nuclear lamina staining when imaging in the 646 nm TA-splitHalo channel again reflecting the recruitment of nHalo and cHalo to the nuclear lamina. This time in cells that do not already contain TA-splitHalo fusions. The scale bar represents 251 μm. (I) Bar graph showing qPCR data validating the presence of ALFA-LMNA and SpyT-LMNA KIs. Internal primers amplified a LMNA specific PCR product in controls and KI cell lines from the AS04 and WT experiments (left). ALFA-LMNA and SpyT-LMNA amplicons were only enriched with respect to the LMNA-internal PCR product in the KI cell populations from both experiments (center and right).

FIG. 5A-I. Tag assisted splitHalo supports allelic multiplexing. FIG. 5A. Overview of knock-in strategy for TA-splitHalo detection of self-associating lamin A/C chains. Knock-In is performed with both ALFA-SpyT and GFP11-SpyT donor strands

(FIG. 5B) Cells that have both of ALFA-SpyT and GFP11-SpyT LMNA edits on different alleles can be sorted and visualized in two-colors when using GFP(1-10) and AS04 TA-splitHalo together (left) or functionalized with both systems of TA-splitHalo (right)

(FIG. 5C) Experimental workflow for TA-splitHalo biallelic sorting in the AS04 cell line. The knock-ins shown in Panel A is performed in the AS04 detection cell line which constitutively expresses NbALFA-nHalo and SpyCatcher-cHalo as well as NLS-TagBFP from a single copy locus. Transfection with GFP(1-10) and staining with 10 nM JF646 yields TA-splitHalo nuclear envelope labelling in two colors each corresponding to a different allele.

(FIG. 5D) Flow cytometry data from the AS04 stable cell line, which constitutively expresses NbALFA-nHalo and SpyCatcher-cHalo as well as TagBFP from a single copy locus. Events (2000 per panel) are shown on log10 scale, and have been gated to show mRuby−, TagBFP+ single cells as described in the methods section. The left panel is a control showing the unedited AS04 cell line. The center panel shows AS04 cells, nucleofected with Cas9 complexed with sgRNA targeting LMNA and an equimolar mixture of single stranded donor DNA introducing either the ALFA-SpyT tandem tag or the GFP11-SpyT tandem tag. Cells in the top right quadrant of this plot were sorted to enrich for GFP+ AND Halo+ cells. The bar graph shows the percentage of cells above the Halo threshold for WT HEK293Ts, the master cell line, our AS04 cells with no KI, or AS04 cells after Cas9 targeting of LMNA with both ALFA-SpyT and GFP11-SpyT donors. Experiments were repeated in two separate wells (rep1 and rep2) for two distinct short guide RNAs (sgRNA1 and sgRNA2) each targeting LMNA near the start codon.

(FIG. 5E) Representative widefield microscopy images of cells sorted in panel D containing both edits. Two color visualization (top panel) shows the single copy NLS-TagBFP constitutively expressed by the AS04 cell line and nuclear envelope GFP and TA-splitHalo signal derived from proteins translated off separate alleles. Allelic multiplexing (bottom panel) is performed by transfection of GFP(1-10)-nHalo and again shows nuclear envelope labeling in both color channels with an increase in TA-splitHalo signal attributed to cells which contain the GFP(1-10)-nHalo transfection. The scale bar represents 25 μm.

(FIG. 5F) Experimental workflow for TA-splitHalo biallelic sorting in the AS04 cell line. The knock-ins shown in Panel A is performed in WT HEK293Ts. Co-transfection of GFP(1-10) and AS04-BFP plus subsequent staining with 10 nM JF646 yields TA-splitHalo nuclear envelope labelling in two colors each corresponding to a different allele.

(FIG. 5G) Flow cytometry data from WT HEK293Ts transiently transfected with our AS04 BLU plasmid (expressing NbALFA-nHalo and SpyCatcher-cHalo as well as mTagBFP2) as well as a plasmid expressing GFP(1-10). Events (2000 per panel) are shown on log10 scale and have been gated to show singlet cells as described in the methods section. The left panel is a control showing the unedited cells. The center panel shows cells nucleofected with Cas9 complexed with sgRNA targeting LMNA and an equimolar mixture of single stranded donor DNA introducing either the ALFA-SpyT tandem tag or the GFP11-SpyT tandem tag. Cells inside polygon gate were sorted to enrich for GFP+ AND Halo+ cells. The bar graphs shows the percentage of cells for WT HEK293Ts, transfected cells with no KI, or transfected cells after Cas9 targeting of LMNA with both ALFA-SpyT and GFP11-SpyT donors. Experiments were repeated in two separate wells (rep1 and rep2) for two distinct short guide RNAs (sgRNA1 and sgRNA2) each targeting LMNA near the start codon.

(FIG. 5H) Representative widefield microscopy images of cells sorted in panel G containing both edits with both gates shown. In the same sorted cell population, we demonstrate two0-color visualization of multiple LMNA alelles using a GFP(1-10) and AS04-BFP co-transfection (top panel). In the same cell line, we can multiplex the TA-splitHalo systems. We have shown this by performing TA-splitHalo labelling with both GFP/Spy and ALFA/Spy TA-splitHalo systems (center and bottom panels). The scale bar represents 25 μm.

(FIG. 5I) Bar graph showing qPCR data validating the presence of ALFA-SpyT-LMNA and GFP11-SpyT-LMNA KIs. Internal primers amplified a LMNA specific PCR product in all controls and KI cell lines from the AS04 and WT experiments (far left). ALFA-LMNA, GFP11, SpyT-LMNA amplicons were only enriched with respect to the LMNA-internal PCR product in the KI cell populations from both experiments (center left, center right, and far right).

### DETAILED DESCRIPTION OF THE INVENTION

The inventors have discovered a tag-assisted fluorescence complementation system that allows for the detection of a first and a second location in one protein (e.g., for specific target protein detection) or the proximity of a first location in one protein and a second location in a second protein (e.g., detecting protein-protein interaction of the two proteins). The system can comprise fusion of a first and a second orthogonal peptide tag (acting as the first and second location) fused to a target protein. The first and second locations can be on the same target protein or on different target proteins depending on what output is desired. Thus, for example where the two locations are in a single target protein, the first and second peptide tags can be fused to the single target protein and expressed in a cell. The target protein fusion can be detected with two components: (1) a first detection fusion protein comprising a first portion of a split reporter and a first affinity agent that specifically binds to the first peptide tag, and (2) a second detection fusion protein comprising a second portion of the split reporter and a second affinity agent that specifically binds to the second peptide tag. The split reporter is designed such that the first portion of the split reporter and the second portion of the split reporter produce a signal when in proximity and are substantially inactive when separate. Thus signal from the portions in proximity can be detected and distinguished from separate, substantially inactive split reporter portions. As explained further below, the system can also be used to detect proximity of two separate proteins, for example where a first target protein comprises the first peptide tag and the second target protein comprises the second peptide tag and they are detected with the detection fusion proteins as discussed above. Further aspects are detailed herein.

The detection systems described involve forming one or more target protein that is a fusion with one or two peptide tags. The target protein can be any protein expressed in the cell or can be a heterologous target protein. In embodiments in which one target protein is to be monitored, two orthologous peptide tags are fused to the target protein to form a fusion target protein. The target protein of interest can be an intracellular or extracellular (e.g., having an extracellular domain linked to a membrane spanning domain) protein.

The two peptide tags (as well as any additional peptide tags used in the system) will typically be orthogonal, meaning that the affinity agent that binds to one of the peptide tags does not bind to the other (or additional) peptide tags. In other words the two peptide tags are bound by different affinity agents that do not significantly cross react with the other peptide or other proteins in the cell.

When expressed on a single target protein, the first peptide tag and the second peptide tag (“first” and “second” is merely used for convenience to distinguish them from each other) are fused in proximity to each other. This is so that the two portions of the split reporter can be brought into proximity when they are bound via their respective affinity agents to the respective peptide tags. In some embodiments, the first and second peptide tags are linked directly, i.e., without an intervening amino acid. Alternatively, the first and second peptide tags can be linked via a linker. Again because proximity of the two peptide tags is desired, generally the two peptide tags are linked via a short linker, e.g., 15 or fewer intervening amino acids (e.g., 10, 5, or 2 or fewer intervening amino acids). Linkers and peptide tag position in the target protein can be selected to avoid interference with target protein function if desired. The linker can be selected to be flexible, for example having a majority or constructed solely from alanine, glycine and serine.

In some embodiments, the first and second peptide tags are fused to the amino terminal of the target protein. In some embodiments, the first and second peptide tags are fused to the carboxyl terminal of the target protein.

Any peptide tag/affinity agent pair that has specific binding can be used. If desired, one can select unique peptides and screen for affinity agents. However, a number of peptide tag/affinity agent pairs are known and can be conveniently used in the systems and methods described herein. For example, exemplary peptide tag/affinity agents include but are not limited to: SpyT/Spycatcher, ALFA/ NbALFA and/or GFP11/GFP1-10. SpyT is AHIVMVDAYKPTK. See, e.g., Zakeri et al., Proc Natl Acad Sci USA 2012 109:E690-697. Alternatively SpyT002 (VPTIVMVDAYKRYK)/Spycatcher002 can be used. See, e.g., Keeble et al., Angell). Chem. Int. Ed. 2017, 56, 16521-16525. The SpyCatcher amino acid sequence is AMVTTLSGLSGEQGPSGDMTTEEDSATHIKFSKRDEDGRELAGATMELRDSSGKTIS TWISDGQVKDFYLYPGKYTFVETAAPDGYEVATAITFTVNEQGQVTVNGKATKGDA HI. The SpyCatcher002 amino acid sequence is AMVTTLSGLSGEQGPSGDMTTEEDSATHIKFSKRDEDGRELAGATMELRDSSGKTIS TWISDGHVKDFYLYPGKYTFVETAAPDGYEVATAITFTVNEQGQVTVNGEATKGDA HT. The ALFA (SRLEEELRRRLTE)/NbALFA system is described in, e.g., Götzke et al., Nature Communications volume 10, Article number: 4403 (2019). NbALFA can be obtained commercially from, e.g., NanoTag Biotechnologies.

GFP11/GFP1-10 refers to a split GFP protein wherein GFP1 is RDHMVLHEYVNAAGIT. GFP 1-10 is a GFP fragment, which contains the three residues that constitute the GFP chromophore, is non-fluorescent by itself because chromophore maturation requires the conserved E222 residue located on GFP11. GFP11/GFP1-10 is described in e.g., Kamiyama, et al., Nat Commun. 2016; 7:11046. GFP11 acts as a peptide tag and GFP1-10 acts as an affinity agent for GFP11, and the combination of GFP11/GFP1-10 results in a further reporter wherein proximity between the two generate fluorescent signal.

The peptide tags, and their proximity in or on a cell, can be detected using two different detection fusion proteins. The detection fusion proteins are (i) a first detection fusion protein comprising an affinity agent that specifically binds to the first peptide tag, wherein the affinity agent is fused to a first portion of a split reporter and (ii) a second detection fusion protein comprising an affinity agent that specifically binds to the second peptide tag, wherein the affinity agent is fused to a second portion of a split reporter. The fusion of an affinity agent and a split reporter can be a direct fusion (without an intervening amino acid sequence) or the affinity agent and reporter amino acid sequences can be linked by an amino acid linker sequence, e.g., 15 or fewer amino acids (e.g., 10, 5, or 2 or fewer amino acids), that does not significantly affect the functions of the affinity agent or split reporter.

The relative position of the affinity agent and the split reporter can be selected so that the affinity agent and split reporter function as desired. Split reporters are composed of an amino-terminal portion of a reporter and a carboxyl portion of the reporter. Each portion of the split reporter is fused to a different affinity agent, as explained herein. In some embodiments, a first affinity agent (which might be for example, SpyCatcher, SpyCatcher002, NbALFA or GFP-1-10) is fused to the amino portion of the split reporter, such that the first affinity agent is at the amino terminus of the fusion and the amino portion of the split reporter (for example, but not limited to the amino-terminal portion of HaloTag) is at the carboxyl terminus of the fusion. Such a fusion can be matched with a second fusion composed of the carboxyl portion of the split reporter (for example, but not limited to the carboxyl-terminal portion of HaloTag) and a second affinity agent (which might be for example, SpyCatcher, SpyCatcher002, NbALFA or GFP-1-10, but is different that the first affinity agent), such that the affinity agent is at the carboxyl terminus of the fusion and the carboxyl portion of the split reporter is at the amino terminus of the fusion. Other relative positions of respective affinity agents and reporter portions in an affinity agent/split reporter pair can also be used.

Split reporters are formed by two reporter portions, which when in proximity due to their linkage to affinity agents than bind adjacent peptide tags, generate a signal (which may optionally also require a substrate). An exemplary signal can be for example fluorescence. Exemplary split reporters can include but are not limited to HaloTag, green fluorescent protein (GFP), Venus, Cre recombinase, Cas9, TEV protease, luciferase, β-galactosidase, esterase or UnaG.

HaloTag refers to a 297 amino acid protein (33 kDa) derived from a Rhodococcus rhodochrous, enzyme having haloalkane dehalogenase activity and where Phe272 is substituted by His272 and designed to covalently bind to a synthetic ligand. See, e.g., Los et al., CS Chem. Biol. 2008, 3, 6, 373-382. During the interaction of the enzyme and ligand, an alkyl-enzyme intermediate is formed during the nucleophilic displacement of a terminal chloride with Asp106. Normally, His272 would function as a general base in wild-type dehalogenase to catalyze the hydrolysis, thus releasing the enzyme. This reaction is altered in the mutant dehalogenase, as the substituted Phe272 does not catalyze the hydrolysis, thus resulting in a covalent adduct with high stability. See, e.g., England et al., Bioconjug Chem. 2015 Jun. 17; 26(6): 975-986. A variety of ligands are available that generate fluorescent signal. HaloTag can be separated into two portions, which portions when in proximity interact to produce the active Halotag reporter but are inactive when separate. See, e.g., Ishikawa, et al., Protein Engineering, Design and Selection, Volume 25, Issue 12, December 2012, Pages 813-820, which describes several possible positions for splitting Halotag into two portions that are active when together, any of which can be used in the methods described herein. The HaloTag ligands include but are not limited to TMR, Oregon Green®, diAcFAM, JF646 and coumarin ligands (available commercially, for example from Promega) that pass through cellular membranes and that generate detectable fluorescent signal when in contact to an active HaloTag reporter (e.g., when split HaloTag portions are in proximity to each other). Depending on the split reporter and substrate/ligand used, it may be useful to include one or more wash steps to remove background non-specific signal from, for example, substrate/ligand.

Split UnaG, e.g. as described in To et al., Protein Sci. (2016) 25:748-753, can be used as a split reporter and can become fluorescent when activated by complementation and subsequent binding of the ligand bilirubin. Bilirubin is naturally present in many cells and can also be added to the sample exogenously. Split UnaG is similar in many aspects to split HaloTag, except that its complementation can be reversed.

Split Cre-recombinase, e.g. as described in Jullien et al., Nucleic Acids Res (2003) 31:e131 can be used as a split reporter and can trigger recombination events at specific DNA sequences when activated by complementation. These recombination events can lead to the alteration of transcription activity or the expressed protein of a reporter gene, which can be subsequently detected either by sequencing of the DNA sequence or by signal from the expression of reporter genes.

Split Cas9, e.g. as described in Zetche et al., Nature Biotechnology (2015) 33:139-142 can be used as a split reporter and can bind and optionally cleave specific DNA sequences when activated by complementation. The binding and/or cleavage events can be coupled to the alteration of the target DNA sequence or modulation of transcription activity, which can be subsequently detected either by sequencing of the DNA sequence or by signal from the expression of reporter genes.

Split TEV protease, e.g. as described in Wehr et al., Nature Methods (2006) 3:985-993, and other split proteases, can be used as a split reporter and can cleave specific peptide sequences when activated by complementation. Its activity can be read out fluorescently when coupled to a fluorescent reporter, e.g. FlipGFP in Zhang et al., J Am Chem Soc (2019)141:4526-4530. It can be coupled to other protease-controlled systems to produce more complicated readouts, e.g. Gao et al., Science (2018) 361:1252-1258.

Split luciferase, e.g. as summarized in Azad et al., Anal Bioanal Chem (2014) 406:5541-5560 can be used as a split reporter and can produce light signal via bioluminescence when activated by complementation and with the presence of enzyme substrate.

Split β-galactosidase, e.g. as summarized in Broome et al., Mol. Pharm (2010) 6:60-74 can be used as a split reporter and can catalyze the hydrolysis of hydrolyze disaccharides such as (β-galactosides when activated by complementation. This chemical reaction can be coupled to the “uncaging” or a caged fluorescent reporter so that the reporter produces fluorescence signal.

Split esterase, e.g. as described in Jones et al., ACS Central Science (2019) 5:1768-1776 can be used as a split reporter and can catalyze the hydrolysis of an ester bond when activated by complementation. This chemical reaction can be coupled to the “uncaging” or a caged fluorescent reporter so that the reporter produces fluorescence signal.

Any fusion proteins described herein can be made as desired. In many embodiments the fusion proteins are encoded by a polynucleotide encoding the fusion protein and then expressed in a cell, e.g., under the control of an operably linked promoter. Nucleic acids encoding the polypeptide fusions can be obtained using routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999). Such nucleic acids may also be obtained through in vitro amplification methods such as those described herein and in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

The affinity agent/split reporters can be used in combination to detect two tags on a single protein, thereby detecting that single protein, or the affinity agent/split reporters can be used to detect proximity of two separate proteins (protein-protein interaction). In either case, the affinity agent/split reporters function by binding to respective peptide tags and when those peptide tags are in proximity (in a single protein or due to interaction of two proteins, each of which have one protein tag) result in bringing the split reporters into proximity, thereby generating a functional enzyme with detectable signal. Accordingly, in some embodiments, the target protein is a single protein and is fused to two different peptide tags. In other embodiments, two target proteins each include a single peptide tag. In yet further variants, one can monitor multiple (e.g., two) different target proteins using different affinity agent/split reporters. For example a first target protein can be fused to a first and second peptide tag and be monitored by a first affinity agent/split reporter pair that bind to that first and second peptide tag, and a second target protein can be fused to a third and fourth peptide tag and be monitored by a second affinity agent/split reporter pair that bind to the third and fourth peptide tag. In some of these embodiments, the first and second target proteins are different amino acid sequences.

In some embodiments, the first and second target proteins are the same or substantially (e.g., at least 90%, 95 or 99% identical) the same amino acid sequence. In this latter embodiment, coding sequences for the first and second target proteins (as fusions with respective peptide tags) can be inserted in different chromosomes, optionally in the same gene in sister chromosomes, and biallelic expression of the two target proteins can be monitored independently in view of the separate affinity agent/split reporter pairs, whose signals are distinguishable. In some aspects, one target protein can be monitored with a first affinity agent/split reporter pair and the second protein target can be monitored with GFP1-10, where the second protein target is fused with GFP 11, wherein signal of the first affinity agent/split reporter pair and intact GFP (i.e., GFP11 and GFP1-10 in proximity) are different.

The fusion proteins can be introduced into cells as desired. Generally, a polynucleotide encoding the target protein fused to the one or two protein tags is introduced into a plurality of cells, where at least some of the plurality of cells express the target protein fusion. Any method of introducing a polynucleotide into a cell of protein expression can be used. Exemplary methods include but are not limited to electroporation or transformation. In some embodiments, a polynucleotide encoding the target protein fusion is introduced into the genome of the cells by introducing a double-stranded break and then introducing the polynucleotide into the break by homologous or non-homologous recombination. A number of technologies have been developed to create double stranded breaks at specific sites including synthetic zinc finger nucleases (ZFNs), transcription activator-like endonucleases (TALENs) and most recently the clustered regularly interspaced short palindromic repeats (CRISPR)/CRISPR-associated protein 9 (Cas9) system.

In some embodiments, the target protein fusion polynucleotide is introduced into the genome of the cells and polynucleotides encoding the affinity agent/split reporters are also introduced into the cell, either as a plasmid or into the genome of the cells. Alternatively, in some embodiments, the target protein fusion polynucleotide is introduced into the genome of the cells and the affinity agent/split reporters are introduced into the cells as proteins. For example, the proteins can be injected, electroporated into the cells, or introduced via other methods (for example, the affinity agent/split reporters can be fused to a polyarginine or other sequence to enable the proteins to pass through the cell membranes). In some embodiments, polynucleotides encoding the affinity agent/split reporters are also introduced into the cells, either as a plasmid or into the genome of the cells, wherein the cells do not (yet) comprise or express the target protein fusion polynucleotide. This latter embodiment can be used by an end user for any desired target fusion protein, which the end user can select and introduce themselves.

Any type of cells can be used in the methods described herein. In some embodiments, the cells are animal cells. For example in some embodiments, the cells are mammalian cells. Exemplary mammalian cells include but are not limited to human, mouse, rat, bovine or porcine cells. In some embodiments, the cells are insect cells. In some embodiments, the cells are plant cells. In some embodiments, the cells are fungal cells. In some embodiments, the cells are prokaryotic cells. In some embodiments, the cells are primary cells, e.g., cells from an individual human or mammal. In some embodiments the cells are cultured cells.

Once the target fusion protein(s) and the affinity agent/split reporters have been introduced into a plurality of cells, expression of the target fusion proteins can be monitored by detection the split reporter signal. The split reporter signal will depend on the split reporter used. In some embodiments, a ligand or substrate of the split reporter is provided so that the split reporter activity can be measured. In some embodiments, the signal is fluorescence. In this case fluorescence can be measured using any instrument useful for measuring fluorescence in cells, including but not limited to FACS instruments, spectrofluorometers (e.g., plate or microplate readers), image cytometers and microscopes. In some embodiments, the signal is bioluminescence (e.g., when the split reporter is a luciferase). In some embodiments, the signal is an alteration of gene or gene expression (e.g. when the split reporter is split Cre recombinase or split Cas9). In some embodiments, the quantity of measured signal can be used to estimate the quantity of expression of the target fusion protein in one or more cell. For example, in some embodiments, the amount of signal is proportional to amount of target fusion protein expressed. In embodiments in which protein-protein interaction is measured by measuring interaction of two fusion proteins each having one peptide tag, the signal measured can be proportional to the interaction of the two target proteins. In some embodiments, the methods can comprise enriching a cell population for cells expressing the target fusion protein. This can be achieved for example using a FACS.

### Examples

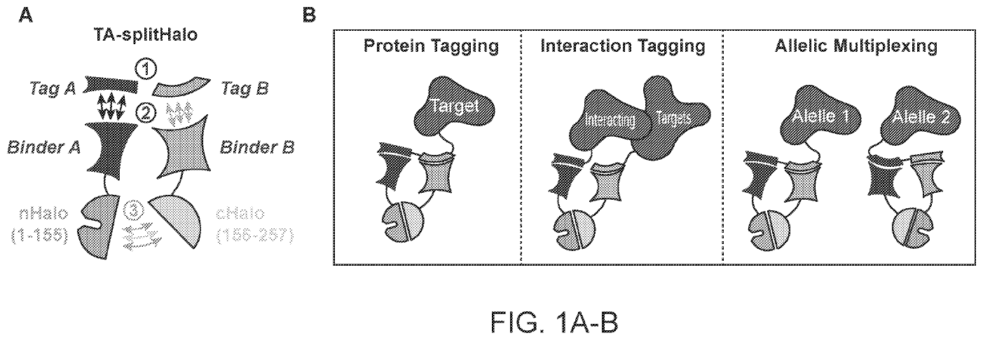

Here, we present a general approach that enables short peptide tagging of proteins to activate split protein complementation, which we named tag-assisted split enzyme complementation (TASEC). For our model system, we focused on HaloTag, a self-labelling enzyme engineered to covalently bind chloroalkane ligands. This property makes HaloTag extremely versatile as available ligands for HaloTag include a range of “turn-on” fluorescent dyes with distinct spectral properties [Grimm, J. B. et al., Super-Resolution Microscopy (ed. Erfle, H.) vol. 1663 179-188 (Springer New York, 2017)] and dyes optimized for single molecule tracking [Grimm, J. B. et al., Nat Methods 13,985-988 (2016)], super-resolution microscopy [Zheng, Q. et al., ACS Cent Sci 5,1602-1613 (2019)], and expansion microscopy [Shi, X. et al., BioRxiv (2019) doi:10.1101/687954]. Based on an existing non-self-complementing split HaloTag [Ishikawa, H. et al., Protein Engineering Design and Selection 25,813-820 (2012)], we have engineered the tag-assisted split HaloTag (TA-splitHalo) that utilizes two orthogonal, short peptide tags and their respective binders in living cells to scaffold the complementation of HaloTag on the target protein (FIG. 1A). We have demonstrated the versatility of this system in the detection of low expression protein targets, the sorting of biallelic KI cells, and the detection of endogenous protein-protein interactions (FIG. 1B).

Results and Discussion

The Engineering of TA-SplitHalo Systems

A TA-splitHalo system consists of two orthogonal peptide tags and their respective binders, arranged in a way to drive efficient complementation of split HaloTag. To identify the set of tags and binders for TA-splitHalo systems and optimal architectures of, we employed a flow cytometry screening assay to test various combinations and arrangements.

The first system we tested in this manner was the GFP/Spy system. In this case, the tags were GFP₁₁ and SpyTag002 (SpyT) and the respective binders were GFP₁₋₁₀ and SpyCatcher002 (SpyC). There are 8 possible TA-splitHalo “architectures” when the HaloTag fragments are positioned at the N- or C-terminus of the two peptide binders. In the GFP/Spy case, we named these architectures GS01 to GS08 . Numerical nomenclature is standardized for all splitHalo architectures where the SpyC and positioning of splitHalo components are the same for each numbered construct.

In an ideal architecture, the TA-splitHalo fragments should only fold if the detector components are expressed and bound to a tagged target. We cloned all 8 possible detector architectures into a common “landing pad” backbone to create a split-Halo detection plasmid library. Since both fusion proteins are expressed on the same plasmid backbone with the same promoter, we can assume the same range of splitHalo fusion protein expression levels relative to one another. Additionally, this vector gives us the ability to generate single-copy cell lines of optimal architectures for subsequent studies.

To rank the architectures, we used GFP₁₁-SpyT-mCherry as the bait, with mCherry giving a readout of tag expression. SpyT-mCherry was used as the negative control for the complementation specificity. In all experiments we used JF646, a far-red HaloTag dye, to avoid sources of cellular autofluorescence and therefore maximize the signal to background ratio.

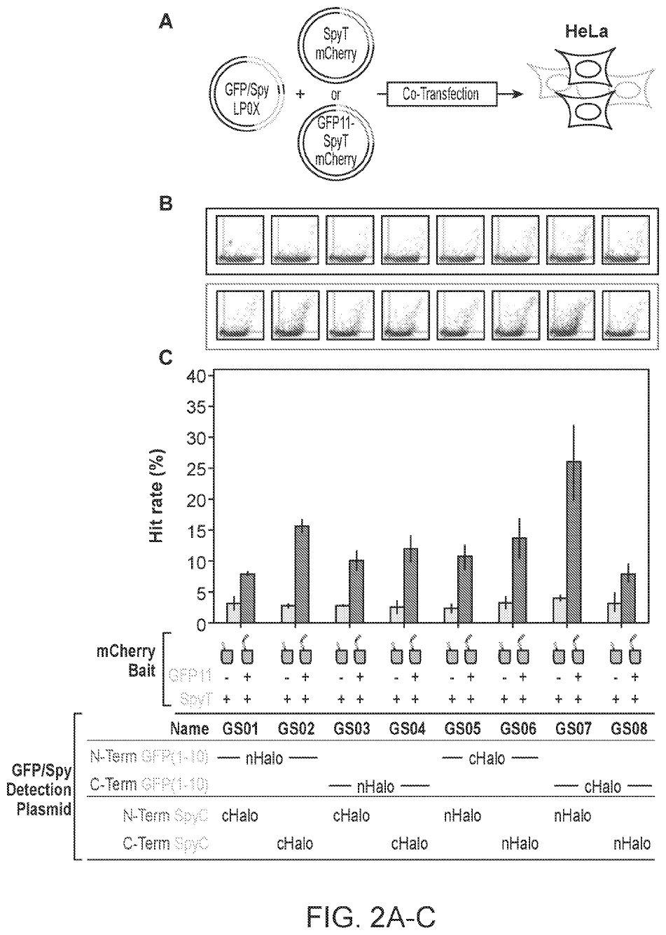

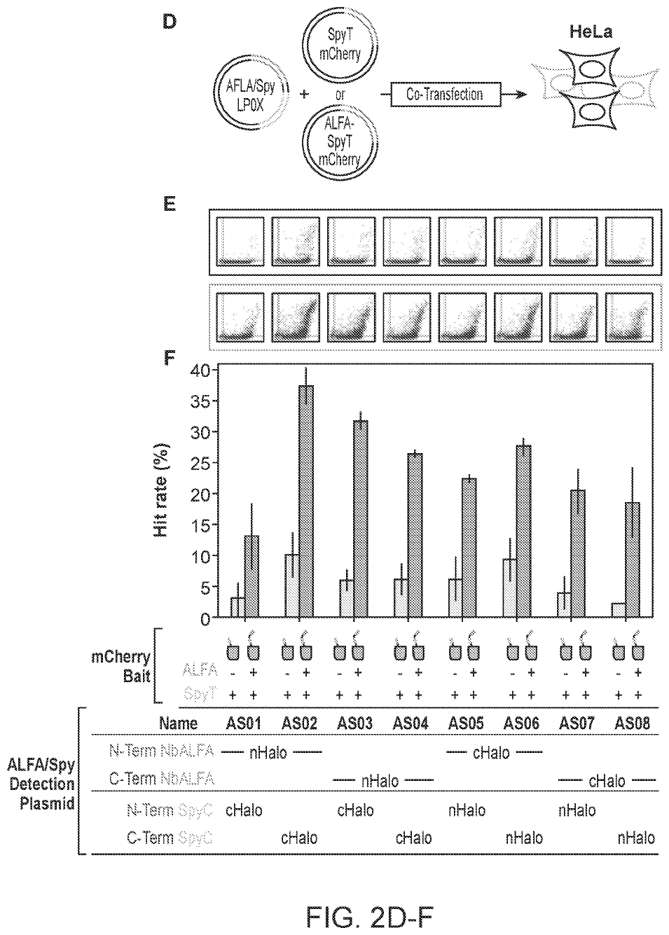

We tested the GFP/Spy system by transfecting each detection plasmid alongside bait plasmids expressing either SpyT-mCherry or GFP₁₁-SpyT-mCherry (FIG. 2A). This was performed in an equimolar ratio with an equivalent number of cells per sample to minimize expression level variability. We developed Python tools to uniformly select singlet cell events and subsequently analyze the relationship between mCherry expression and reconstitution-derived splitHalo. From the raw data (FIG. 2B), we obtained hit rates (Halo+/mCherry+) for each architecture (FIG. 2C). Analysis shows that all GFP/Spy architectures impart statistically significant conditionality for the condition with both tags as hypothesized. However, there are differences when it comes to true hit rate across different architectures. We picked GS02 and GS07 for further characterization. GS02 has greatest fold difference between the background hit rate and the true hit rate. Conversely, GS07 yields the highest splitHalo signal when both tags are present, but it also has the second most background of any GFP/Spy architecture.

The next system we tested was ALFA/Spy splitHalo. The ALFA tag is a structured α-helix peptide with a cognate nanobody named NbALFA that we employed as the binder [Götzke, H. et al., Nat Commun 10, 4403 (2019)]. We held SpyT constant to make direct comparisons between various splitHalo systems. The ALFA/Spy system is dark until the addition of a Halo ligand because there are no extraneous fluorophores in the architectures.

We employed the same screening strategy to test the ALFA/Spy architectures, AS01 to AS08. In this case, we transfected each detection plasmid alongside bait plasmids expressing either SpyT-mCherry or ALFA-SpyT-mCherry in an equimolar ratio (FIG. 2D). Again, the raw data (FIG. 2E) were analyzed, and architecture-specific hit rates were obtained (FIG. 2F). Like the GFP/Spy system, all architectures exhibited with statistically significant signal increases with two tags aside from AS01. From the ALFA/Spy system, we selected AS02 and AS04 for further study. AS02 has the highest hit rate of the ALFA/Spy architectures while AS04 is another architecture that performed well with the SpyC-cHalo component. This allowed us to attribute the differences seen when comparing GS02, AS02, and AS04 solely to the varied nHalo fusion.

To further investigate the specificity of our four best performing architectures, we repeated our assay, adding an untagged mCherry bait to determine whether splitHalo background in the SpyT controls was the result of SpyT recruitment. The results from this test show that while co-transfection alongside SpyT-mCherry resulted in a slightly increased hit-rate in most architectures over untagged mCherry, the results were not statistically significant from co-transfection with untagged mCherry. This means that any background we see is likely from highly transfected cells.

Detection of Knock-In Cells using TA-SplitHalo

To determine the utility of splitHalo systems for detecting successful KI events, we generated stable HEK293T cell lines to allow fair comparison between our selected split-Halo architectures and against the legacy split GFP_{1-10/11} platform. Via BxbI-driven integration, we created cell lines with single-copy integrants of the four selected split-Halo detection architectures. For comparison, we also created a GFP₁₋₁₀ cell line in a similar manner. By placing the detection modules at the same genomic site—the AAVS1 safe harbor locus—we can compare the split-Halo systems to the split-GFP with the detection proteins present in the cell lines in known relative quantities. This way, we can compare KIs to the same target across all the cell lines and KI strategies.

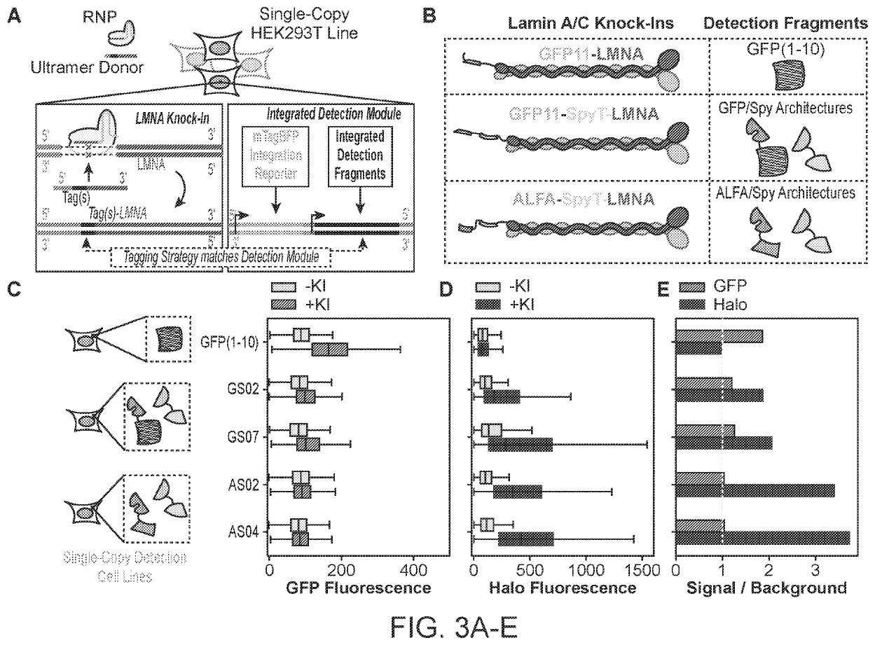

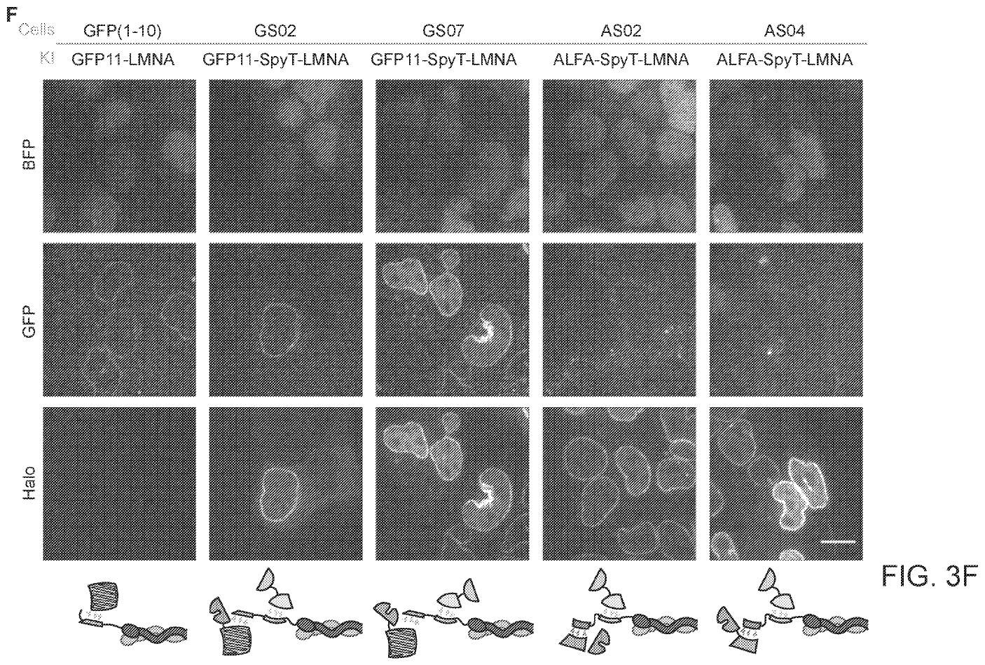

After generating and validating the landing pad cell lines through genomic PCR, we performed a KI targeting the LMNA gene in each cell line with the tagging strategy that corresponds with each detection module (FIG. 3A-B). After sorting these KIs, we characterized the KI populations using the original isogenic lines as controls through Python analysis (Figure S6). In this manner, we can compare how split-Halo systems and architectures compare in relation to one another and GFP_{1-10/11}. By taking the ratio of the median signal intensities for cell populations of the original cell lines and sorted LMNA KI lines in the GFP and splitHalo channels (FIG. 3C-3D), we can compare signal to background ratios in each channel for the same KI target across detection platform (FIG. 3E).

After performing this analysis, we can see that the GFP_{1-10/11} system yields a 1.85 signal to background ratio in the landing pad cell line. In comparison, each of the TA-splitHalo architectures perform comparably or better in the far-red channel using 10 nM JF646 dye. In the ALFA/Spy architectures, the signal to background ratio was 3.4 and 3.7 for AS02 and AS04 respectively, demonstrating that splitHalo outperforms GFP_{1-10/11} for detection of LMNA KI.

Confocal imaging confirmed that both GFP and splitHalo signal had a nuclear envelope localization corresponding to lamin (FIG. 3E). When we set laser power and contrast levels at the same level in these channels, the imaging data independently verifies that TA-splitHalo outperforms GFP_{1-10/11}-based systems in terms of brightness over endogenous background. In the splitHalo systems, most of the background originates from basal levels of tag-independent splitHalo complementation. For architectures with high median background like GS07, we see that this corresponds to visible cytoplasmic TA-splitHalo signal verifying that this unwanted signal is not driven by any single tag and is the result of non-specific complementation.

Detecting protein-protein interactions with TA-splitHalo

After quantifying the performance of the strategy in a knock-in setting, we sought to test whether the TA-splitHalo strategy allowed us to enrich for cells containing a protein-protein interaction. Because TA-splitHalo tagging systems consist of two peptide tags, we tested whether separating the tags and placing them on interacting proteins could yield a sortable signal upon complex formation or multimerization.

For this purpose, we used the homodimerization of lamin A/C chains as a model system, which places the N-termini of separate monomers in proximity [Dittmer, T. A. & Misteli, T., Genome Biol 12, 222 (2011); Ahn, J. et al., Nature Communications 10, 3757 (2019)]. We expected to see complemented Halo signal when the two tags of TA-splitHalo are present on different alleles of the LMNA gene.

Specifically, we modified our LMNA KI protocol to include two ultramer donor strands in the AS04 cell line, so that we can achieve simultaneous double-KI of ALFA-LMNA and SpyT-LMNA (FIG. 4A) leading to TA-splitHalo complementation at lamin dimers (FIG. 4B). Once we perform the KI and stain with JF646, Halo+ cells should contain both edits (FIG. 4C). We enriched for this Halo+ population (FIG. 4D) and confirmed nuclear envelope labelling using widefield imaging (FIG. 4E).

Having demonstrated that our splitHalo system allows protein-protein interaction sorting in a dedicated cell line, we wanted to show that this could be achieved in a wild type background. For this purpose, we designed a reporter strategy to eliminate the high transfectants that are the source of background as seen in data from our architecture benchmarking (FIG. 2). A compatible reporter would enable real-time screening and eliminate high transfectants on the FACS machine even when dampening expression by reducing the amount of transfected plasmid fails to account for all the background cells. To this end, we cloned splitHalo-BFP plasmids for our selected architectures. In these plasmids, we added an mTagBFP2 reporter. mTagBFP2 has a spectral emission that does not overlap with that of JF646 and thus allows us to better sort true splitHalo positive cells.

When we performed the ALFA-LMNA + SpyT-LMNA sort in WT HEK293Ts, we transfected the AS04-BFP plasmid and set a gate on a range of BFP expression values where there is minimal Halo background in the no KI transfection control (FIG. 4F). In the KI populations, we see a significant increase in Halo+ cells that mirrors our landing pad results (FIG. 4G). Constraining the population of interest to cells that are minimally transfected emulates the landing pad cell line where there is only one copy of the TA-splitHalo transcriptional units. Again, we see that this signal is specific to lamin in widefield images of the sorted cells (FIG. 4H).

To confirm that we are enriching cells with LMNA edits, we performed RT-qPCR on cDNA derived from RNA extracted from sorted KI and control populations for this and subsequent experiments. We used four primer pairs on each sample including one LMNA internal control and three to distinguish edited ALFA, SpyT, and GFP₁₁ LMNA-specific edits. Compared to all controls without KIs including wild-type HEK293Ts, the parent landing pad cell line and AS04 cells, we confirmed that KI sorts enrich for both ALFA-LMNA and SpyT-LMNA KIs (FIG. 4I).

We also demonstrated that TA-splitHalo can also detect interactions between two different proteins. Using the AS04 detector cell line, we performed double KI on 7 pairs of proteins known to interact with each other, including LMNA and heterochromatin protein 1 (HP1, also named as CBXS) (Ye, Q.; Worman, H. J. J Biol Chem 1996, 271 (25), 14653-14656.), myc-associated factor X (MAX) and MAX dimerization protein 1 (MXD1) (Grandori, C.; et al. Annu Rev Cell Dev Blot 2000, 16, 653-699), MAX and the transcription factor c-Myc (MYC) Grandori, C.; et al. Annu Rev Cell Dev Biol 2000, 16, 653-699), proteasome activator PSME4 and proteasome α-subunit PSMA3 (Guan, H., et al., PLoS Biol 2020, 18 (3), e3000654), PSME4 and proteasome β-subunit PSMB2 (Guan, H., et al , PLoS Biol 2020, 18 (3), e3000654), cohesin subunits RAD21 and SMC1A (Peters, J.-M., et al, Genes Dev 2008, 22 (22), 3089-3114), and microtubule components α- and (β-tubulin (TUBA1B and TUBB4B)(Nogales, E., et al., Nature 1998, 391 (6663), 199-203). In all cases, FACS of KI cells enriched Halo+ cells over the parent AS04 cell line. Confocal images of sorted cells showed signal from the expected subcellular compartments or structures. In particular, TA-splitHalo signal from CBXS/LMNA specifically highlighted the perinuclear region where heterochromatin is in contact with nuclear lamina, despite the presence of 1-1P1 (CBXS) elsewhere in the nucleus (Ye, Q.; Worman, H. J. J Biol Chem 1996, 271 (25), 14653-14656). Similarly, while proteasomes exist throughout the cell, TA-splitHalo signal with PSME4 is enriched in the nucleus because of the nuclear localization of PSME4 (Guan, H., et al., PLoS Biol 2020, 18 (3), e3000654). These observations demonstrated the specificity of TA-splitHalo in detecting protein-protein interactions. We further confirmed that TA-splitHalo does not induce artificial interactions by showing that non-interacting SpyT-mCherry did not mis-localize to the nuclei in MAX/MYC KI cells, which contain accessible ALFA-MAX (free or bound to non-tagged MYC from unedited alleles).

Allelic Multiplexing with TA-SplitHalo

After demonstrating that we can perform a simultaneous KI on multiple alleles, we sought to leverage the multiplexing capabilities of the two splitHalo systems for novel applications in KI enrichment. We aimed to sort cells which are GFP/Spy and ALFA/Spy TA-splitHalo compatible on the same target gene. Currently, isolating biallelic KI populations while retaining identical functionality on both loci is difficult to do without extensive clonal verification. In our special case, the dependence of the GFP/Spy system on split GFP_{1-10/11} allows us to sort the GFP₁₁-SpyT KI using a traditional split GFP_{1-10/11} workflow. Thus, when we KI both GFP₁₁-SpyT and ALFA-SpyT to the same gene in the same cells (FIG. 5A), we can sort for each edit in a different color channel (GFP and splitHalo+ JF646 respectively), yielding cells in which TA-splitHalo can be recruited to proteins translated off multiple alleles of the same gene (FIG. 5B).

Like with the protein-protein interaction sorts, we first performed this sort using the AS04 landing pad. Our KI protocol included two ultramer donors, one containing GFP₁₁-SpyT and the other containing ALFA-SpyT. The AS04 landing pad already contains the detection components for the ALFA/Spy TA-splitHalo system at optimal concentrations, so in this case GFP₁₋₁₀ was the only transfection needed for the two-color biallelic sort. Cells that are GFP+ and Halo+ should have both KIs on separate alleles of the LMNA gene (FIG. 5C). When we exclude cells that lack the integrated detection fragments, we see an enrichment of Halo+ GFP+ cells in the KI population (FIG. 5D). After sorting this population, we demonstrated that we can multiplex both splitHalo systems by transfecting GFP₁₋₁₀-nHalo. This completes the pair of fusions necessary as the GS02 splitHalo architecture is in a cell line engineered to contain the AS04 components. In cells which take up the plasmid, we expect to see nuclear envelope signal in both colors corresponding to the two independent edits and an increase in splitHalo signal due to the presence of both TA-splitHalo systems. Our widefield images confirm the expected split GFP_{1-10/11} and TA-splitHalo lamin signal and show a clear increase in signal in transfected cells (FIG. 5E). As with the protein-protein interaction experiments, we grew the enriched cells from each population. We generated cDNA from these samples and analyzed the relative fraction of edits in each population by qPCR. These results confirm enrichment of all three tags in these cells compared to pertinent controls (FIG. 5F).

We also performed similar KIs on LMNA in wild-type HEK293Ts. In this case we transfected the pre-sorted cells containing KIs with GFP₁₋₁₀ and AS04-BFP. GFP₁₋₁₀ was used to sort any GFP+ cells containing the GFP₁₁-SpyTag KI while AS04-BFP is used to sort and Halo+ cells containing the ALFA-SpyT KI (FIG. 5G). To sort the population with both edits, we used a “true-splitHalo positive” gate to take into account the proportional increases of TA-splitHalo signal in high transfectants and a nested gate to sort for GFP+ cells (FIG. 5G). Widefield imaging shows that we can use GFP/Spy and ALFA/Spy TA-splitHalo systems in this population as well as visualize protein from both alleles using the same transfection we used to sort (FIG. 5H). Again, qPCR validated that we are sorting cells with all three tags enriched (FIG. 5I).

TA-SplitHalo Exemplifies and Enables TASEC Approaches

In this work, we introduce TASEC, a technique that employs short peptide tags to recruit and refold split enzymes, enabling complex interfacing with target proteins with minimal scarring. Specifically, we illustrate how to engineer a TASEC system and leverage its strengths in CRISPR/Cas9-mediated KIs. The utilization of TASEC in this manner enables us to reconstruct powerful enzymes with desired functions on any endogenous target conditional upon a specific genetic edit. Here, we applied this strategy to develop TA-splitHalo.

TA-splitHalo is a platform which proved to be an optimal system to demonstrate the strengths of the generalizable TASEC approach. It is a scalable platform that expands our capabilities for enriching KI cells and generates versatile cell lines that can exploit the full suite of HaloTag applications. Additionally, TA-splitHalo offers a rapid, non-destructive method to select and validate tandem tagged KI cells. These cell lines could then be used for architecture tests of any TASEC system. For example, Renilla Luciferase has ˜35% homology to the HaloTag and its split may be interchangeable with splitHalo once a successful TA-splitHalo system has been identified [Paulmurugan, R. & Gambhir, S. S., Anal Chem 75, 1584-1589 (2003)]. Other existing split enzymes that could be tested as TASEC systems in tandem tagged cell lines include split-TEV protease [Wehr, M. C. et al., Nat Methods 3, 985-993 (2006)], split-Cre recombinase [Hirrlinger, J. et al., PLoS ONE 4, e4286 (2009)], split-Firefly luciferase [Paulmurugan, R., Umezawa, Y. & Gambhir, S. S. Proc Natl Acad Sci USA 99, 15608-15613 (2002)], split-DamID [Hass, M. R. et al., Mol Cell 59, 685-697 (2015)] and split-esterase [Jones, K. A. et al., ACS Cent Sci 5, 1768-1776 (2019)]. Though we have optimized the system in human cell lines, the TA-splitHalo systems we describe should be applicable in model systems across all three kingdoms (eukaryotes, prokaryotes, and archaea).

The flow cytometry-based approach we used to decipher working TASEC architectures is applicable to any split enzyme with a fluorescence readout. From this approach, we derived two different TA-splitHalo systems from our architecture scanning that yield unique benefits. The GFP/Spy TA-splitHalo system incorporates a split-FP as one of the tag/binder pairs. This can be used to increase stringency while sorting and allows for the ability to visualize or track endogenous targets when using non-fluorescent HaloTag ligands. The ALFA/Spy TA-splitHalo system provides a way to recruit splitHalo with no extraneous fluorophores. The system yields “turn-on” Halo-tag fluorescence where cells remain dark in all channels even after full complementation of the ALFA/Spy architecture. ALFA Tag and NbALFA mutants are also excellent templates for developing orthogonal mutants and further multiplexing capabilities without the use of splitFPs that restrict applications in specific color channels.

TA-splitHalo Expands Utlity of CRISPR/Cas Knock-In Methods

Our demonstration of detecting a protein-protein interaction using TA-splitHalo provides an example of how TASEC systems can be used to study relationships between endogenous molecules. For investigating characterized interaction partners, TA-splitHalo provides a way to translate these studies into environments with high autofluorescence like organoids, embryos, and animal models due to the possibility to use long wavelength dyes [Heppert, J. K. et al., MBoC 27, 3385-3394 (2016)]. By varying the concentration of Halo dye, TA-splitHalo could be used to study protein-protein interactions at the single molecule level, with limiting dye, or at the macro level, with saturating dye. When screening for unknown interaction partners, splitHalo can be used in an unbiased screen to sort, validate, and possibly purify interaction partners. In the future, we can look to place a pair of TASEC tags on adaptor proteins that bind to specific DNA and RNA sequences like noncutting variants of Cas9 [Chen, B. et al., Cell 155, 1479-1491 (2013)] and Cas13[Abudayyeh, O. O. et al., Nature 550, 280-284 (2017)] respectively. In this way, we can generate TASEC functionality driven by the presence of specific DNA or RNA sequences.

We have also shown that TA-splitHalo enables the sorting of complex populations by isolating biallelic KIs using multiplexing of the two TA-splitHalo systems. Employing both splitHalo systems simultaneously in a single round of FACS, we have bypassed the clonal selection and multiple genotyping steps that traditionally made this process laborious. Furthermore, if we use TA-splitHalo tagging schemes solely for enrichment, other functional sequences of interest can be added to each donor strand. Resulting cell lines therefore would contain either the same KIs on multiple alleles or varied KIs on each allele. This is a particularly important advance when tagging both alleles of a gene with a protein or peptide tag that is not detectable via FACS sorting. Additionally, the ability to sort cells with KIs on both alleles allows for manipulation of each allele separately or together in the same cell line through RNAi or protein fusions containing the TA-splitHalo binders. This is important for applications where there may be differences between perturbing one allele or both alleles. Ability to sort biallelic KIs in this fashion also empowers studying patient-derived cellular models of genetic disease where one allele is altered and behaves differently than the other. Finally, methods to separate genetically modified cells by number of alleles edited will be an important quality control for cell therapies in the future [Roth, T. L Curr Hematol Malig Rep 15, 235-240 (2020)].

While TA-splitHalo is a notable advance for high-throughput sorts of complicated KI populations, a key feature is that the library of compatible ligands maximizes the potential of the sorted cell lines. Since the splitHalo ligand and saturation level can be decided on after a KI occurs and just prior to any application, the most appropriate ligand can be strategically selected each time the TA-splitHalo system is employed in the same cell line. For example, in protein labelling applications, TA-splitHalo is the first platform that outperforms the background adjusted brightness of GFP while also retaining the cost-effective workflows of splitFPs when using JF646. This property should allow a wider range of the human proteome to be sorted and imaged. Halo dyes in other channels can be selected to work around other fluorophores. This attribute would be valuable for flexibility in multicolor flow cytometry panels and imaging experiments. Finally, the library of available HaloTag ligands also includes molecules to facilitate purification [Méndez, J. L. et al., BioTechniques 51, 276-277 (2011)] and degradation [Tovell, H. et al., ACS Chem. Biol. 14, 882-892 (2019); Simpson, L. M. et al., Cell Chemical Biology 27, 1164-1180.e5 (2020)] of target proteins that widens the range of experiments possible with TA-splitHalo.

In conclusion, TA-splitHalo provides a modular, minimalistic, scalable means to sort traditional or complex KI populations with a growing library of HaloTag ligands, making the system highly versatile. It also provides a blueprint for applying a TASEC approach to CRISPR/Cas9-mediated KIs and a path to onboarding new TASEC systems that can generate custom readouts linked to expression of native macromolecules or interactions between them with short peptide tags.

Methods

Cloning

We generated part vectors, expression vectors, and landing pad vectors following the Mammalian Toolkit (MTK) approach [Fonseca, J. P. et al., ACS Synth. Biol. 8, 2593-2606 (2019)].

10 μL reactions to generate part vectors consisted of 40 fmol insert DNA clean of BsaI and BsmBI restriction sites, 20 fmol MTK part vector backbone, 10× T4 Ligase Buffer (NEB B0202S), Esp31 (NEB R0734S/L), and T7 DNA Ligase (M0318S/L). The reactions were cycled between digestion at 37° C. for 2 minutes and ligation at 25° C. for 5 minutes. 1 μL of the resulting reaction mixture was transformed into MachI E. coli (QB3 Macrolab) and colonies lacking GFP expression were selected for amplification and sequencing verification.

To streamline cloning of the expression vectors, transcriptional unit-specific CDS backbones were generated by adding the requisite connector sequences, a PGK promoter, a BGH terminator and poly(A) to the original MTK assembly backbone, also known as pYTK095 (Addgene #65202). With these backbones, we improved workflows by reducing the number of inserts needed to generate new assemblies. Expression vectors were generated in 10 μL reactions containing 20 fmol CDS backbone, 40 fmol of each part insert, 10× T4 Ligase Buffer, BsaI-HF v2.0 (NEB R3733S/L), and T7 DNA Ligase with the same cycling conditions as the part vectors.

Landing pad (LP) vectors were generated similarly to part vectors in 10 μL reactions with 20 fmol MTK landing pad entry backbone (Addgene #123932), 40 fmol of each expression vector plasmids, 10× T4 Ligase Buffer (NEB B0202S), Esp31 (NEB R0734S/L), and T7 DNA Ligase (M0318S/L) with the same cycling conditions as the part vectors. For generating landing pad vectors from expression vectors without the correct overhangs, an oligonucleotide stuffer was used to complete the overhangs.

TA-splitHalo-BFP plasmids were made in 10 μL reactions comprising 20 fmol Kanamycin ColE1 digested backbone, 40 fmol TA-splitHalo fusion expression vectors, 40 fmol PGK-mTagBFP2 expression vector, 10× T4 Ligase Buffer (NEB B0202S), Esp31 (NEB R0734S/L), and T7 DNA Ligase (NEB M0318S/L) with the same cycling conditions as the part vectors.

Transfection of HeLa Cells in 8-Well Chamber Flasks for TA-SplitHalo Architecture Benchmarking

For FIG. 1 transfections, HeLa cells were seeded in an 8-well chamber flask at cells per well in 225 uL DMEM +1% Penicillin/Streptomycin (P/S) 10% Fetal Bovine Serum (FBS). 160 ng of each TA-splitHalo architecture plasmid was cotransfected with 80 ng of mCherry bait plasmid with 0.7 μL FuGENE HD. This corresponded to a 1:1 molar ratio. After an overnight incubation, samples were stained with 10 nM JF646 dye in 100 μL Phenol Red-free DMEM +1% Penicillin/Streptomycin 10% Fetal Bovine Serum. Flow cytometry was performed the day after overnight staining.

Seeding and Transfection of HEK293T KIs for TA-SplitHalo Sorting

In all experiments 6-well chamber flasks were seeded with 300k pre-sorted KI cells and controls were seeded in 2 mL of DMEM +1% P/S 10% FBS.

For FIG. 4G, 180 fmol of AS04-BFP plasmid was transfected with 2.8 μL FuGENE HD in each well containing control HEK2993Ts and pre-sorted LMNA KI cells.

For FIG. 5D in AS04 cells, 600 fmol of GFP1-10 plasmid was transfected with 9.3 μL FuGENE HD in each well containing control AS04 cells and pre-sorted LMNA KI AS04 cells.

For FIG. 4G, 600 fmol of GFP1-10 and 180 fmol of AS04-BFP plasmid were cotransfected with 9.3 μL FuGENE HD in each well containing control AS04 cells and pre-sorted LMNA KI AS04 cells.

In all cases, cells were stained in 10 nM JF646 in 1 mL of Phenol Red-free DMEM +1% P/S 10% FBS after an overnight transfection. Cells were FACS sorted the day after staining.

Seeding and Transfecting HEK293Ts for TA-SplitHalo Imaging

In all experiments, 8-well chamber flasks were pre-treated with poly-L-lysine seeding.

For AS04-BFP imaging in FIG. 4H, 20k HEK293T cells containing ALFA-LMNA SpyT-LMNA KIs were seeded in each well. After incubation overnight, 15 fmol AS04-BFP was transfected with 0.7 μL FuGENE HD.

For AS04 cell imaging in FIG. 5E, 20k sorted AS04 cells containing ALFA-LMNA SpyT-LMNA KIs were seeded in each well. After incubation overnight, we performed 50 fmol transfections of GFP1-10 and GFP1-10-nHalo with 0.7 μL FuGENE HD in different wells.

For AS04 cell imaging in FIG. 5E, 20k sorted HEK293T cells containing ALFA-LMNA SpyT-LMNA KIs were seeded in each well. After incubation overnight, we performed 15 fmol GS07-BFP, 15 fmol AS04-BFP, and 50 fmol GFP1-10+15fmol AS04-BFP transfections with 0.7 μL FuGENE HD in different wells.

After each of these transfections, Cells were stained with 10 nM JF646 after an overnight incubation in 100 μL Phenol Red-free DMEM +1% Penicillin/Streptomycin 10% Fetal Bovine Serum and imaged the subsequent day.

Lamin A/C gRNA IVT Template Synthesis

The IVT template for LMNA gRNA was made by PCR. The reactions are done in a 100 μL reaction containing 50 μL 2× Phusion MM (ThermoFischer F531L), 2 μL ML557+558 mix at 50 μM, 0.5 μL ML611 at 4 μM, 0.5 μL of each gene-specific oligo at 4 μM, and 47 μL DEPC H₂O. The PCR product was purified using a Zymo DNA Clean and Concentrator Kit (Zymo Research D4014). Sequences for these primers and thermocycling conditions are given in Figure SX.

Lamin A/C gRNA Synthesis

IVT was carried out using the HiScribe T7 Quick High Yield RNA Synthesis Kit (NEB E2050S) with the addition of RNAsin (Promega N2111). Purification of mRNA was performed using the RNA Clean and Concentrator Kit (Zymo Research R1017). gRNA was stored at −80° C. immediately after measuring concentration and diluting to 130 μM.

Generation of Split-Halo Landing Pad Detection Cell Lines

The split-GFP, and split-Halo Landing Pad HEK293Ts, were generated from a published landing pad parent cell line [Fonseca, J. P. et al., ACS Synth. Biol. 8, 2593-2606 (2019)] seeded at 100k cells in a 12-well plate. To each well, 600 ng of BxbI Integrase Expression Vector (Addgene #51271) and 600 ng of each landing pad donor plasmid were co-transfected. Once cells are confluent, cells were split once and seeded in a T25 flask, and blasticidin (Gemini Bio-Products 400-165P) was added at 51.1 μg/mL for selection prior to FACS sorting integrated cell lines.

Cas9 HDR Knock-Ins

The day prior to performing the KI, 2.5 million HEK293Ts were treated with 200 ng/mL nocodazole and seeded at 250k cells/mL in 10 mL DIMEM media (Sigma-Aldrich M1404) before incubation overnight for 15-18 h prior to nucleofection.

The next day, RNPs were generated in 10 μL reactions consisting of 1 μL sgRNA at 130 μM, 2.5 μL purified Cas9 at 40 μM, 1.5 μL HDR template at 100 μM, 2 μL, 5× Cas9 Buffer, and DEPC H₂O up to 10 μL. HDR template ultramer sequences synthesized from IDT are given in Table S1.

In a sterile PCR or microcentrifuge tube, Cas9 Buffer, DEPC H₂O, and sgRNA were mixed and incubated at 70° C. for 5 min to refold the gRNA. During this step, 10 μL aliquots of purified Cas9 at 40 μM was thawed on ice. Next, 2.5 μL Cas9 protein was slowly added to the diluted sgRNA in Cas9 buffer and incubated at 37° C. for 10 min. Finally, 1.5 μL of each ultramer donor was to the RNP mix and all samples were kept on ice until ready for nucleofection.

For efficient recovery post-KI, a 24-well plate with 1 mL media per well was incubated in a 37° C. An appropriate amount of supplemented Amaxa solution corresponding to the number of KIs to be performed was prepared room temp in the cell culture hood. For each sample 16.4 μL SF solution and 3.6 μL supplement was added to an Eppendorf tube for a total of 20 μL per KI. Amaxa nucleofector instruments/computers were then turned on and kept ready for nucleofection.

Nocodazole-treated cells were harvested into a sterile Falcon tube and counted. A volume equivalent to 200k cells per KI was transferred to another Falcon tube and centrifuged at 500 g for 3 min. Remove supernatant containing nocodazole-treated media and resuspend in 1 mL PBS to wash. The cells were centrifuged again at 500 g for 3 min. PCR tubes containing RNPs were brought into TC hood.

Cells were resuspended in supplemented Amaxa solution at a density of 10k cells/μL. 20 μL of the cell resuspension was added to each 10 μL RNP tube. The cell/RNP mix was pipetted into the bottom of the nucleofection plate. The nucleofection was carried out on a Lonza 96-Well shuttle Device (Lonza AAM-1001S) attached to Lonza 4D Nucleofector Core Unit (Lonza AAF-1002B). Cells were nucleofected using CM-130 program and recovered using 100 μL media from the pre-warmed 24-well plate and transferred to the corresponding well.

Once cells reached 80% confluence in the smaller vessel, they were transferred first to a 6-well plate and then to a T25 flask. Cells were FACS sorted after a week of maintaining and expanding the pre-sorted KI population to reach optimal cell numbers and Cas9-mediated cutting and repair.

Cell Line Genotyping

Genomic DNA was prepared from 1 million cells using the Monarch Genomic DNA Purification Kit (NEB, #T3010G). Diagnostic PCR was then carried out followed by gel extraction (NucleoSpin) and Sanger Sequencing (Quintara Biosciences).

Confocal Imaging

Cells were imaged on a Nikon Ti Microscope equipped with a Yokagawa CSU22 spinning disk confocal and an automated Piezo stage. We used a CO₂- and temperature-controlled incubator it is ideal for live specimen imaging. Our laser lines were 405 nm, 491 nm, 561 nm, 640 nm. Pixel binning was set at 2×2.

Widefield Imaging

All widefield imaging was performed on a Nikon Ti-E microscope equipped with a motorized stage, a Hamamatsu ORCA Flash 4.0 camera, an LED light source (Excelitas X-Cite XLED1), and a 60× CFI Plan Apo IR water immersion objective. All downstream image analysis was performed in ImageJ.

qPCR

Total RNA was extracted from 1 million cells using the Monarch Total RNA Miniprep Kit (NEB, #T2010S). We prepared cDNA from 1 μg of extracted RNA using LunaScript® RT SuperMix Kit (NEB, #E3010). No Template and No Reverse Transcriptase controls (NTC and NRT) were performed in parallel to cDNA preparations. We set up qPCR plates using 0.5 μl of each 20 μl cDNA sample, 10 μl 2× Maxima SYBR Green qPCR Master Mix (Thermo Scientific K0221), and optimized primer pairs corresponding to SpyT-specific, GFP₁₁-specific, and ALFA-specific LMNA KIs. We also ran a primer set specific to the wild-type LMNA gene for a positive control and reference marker.

For standard curves, we cloned plasmids containing sequences corresponding to all edited and unedited versions of the LMNA gene. RT-qPCR was performed on QuantStudio™ 5 Real-Time PCR System. These primer sequences are listed in the Table below.

Flow Cytometry Analysis and Cell Sorting

FACS sorting and flow cytometry was performed on a BD FACSAria II in the Laboratory for Cell Analysis at UCSF. mTagBFP2 signal was measured using the 405 nm laser with a 450/50 bandpass filter, GFP signal was measured with the 488 nm laser and 530/30 bandpass filter, mCherry signal was measured using the 561 nm laser and 610/20 bandpass filter and TA-splitHalo signal was measured with the 633 nm laser with a 710/50 bandpass filter. Files in the .fcs format were exported from the BD FACS Aria II were analyzed in Python using our altFACS package.

TABLE S1

| Ultramer | |
|---|---|
| Name | Ultramer Sequence |
| GFP11-SpyT- | TTTCCGGGACCCCTGCCCCGCGGGCAGCGCTGCCAACCTGC |
| LMNA | CGGCCATGCGTGACCACATGGTCCTTCATGAGTATGTAA |
| | ATGCTGCTGGGATTACAGGTTCTGTGCCTACTATCGTGA |
| | TGGTGGACGCCTACAAGCGTTACAAGGGATCCGAGACC |
| | CCGTCCCAGCGGCGCGCCACCCGCAGCGGGGCGCAGG |
| | CCAGCT |
| ALFA-SpyT-LMNA | CCTTTCCGGGACCCCTGCCCCGCGGGCAGCGCTGCCAACCT |
| | GCCGGCCATGCCTAGCCGCCTGGAGGAAGAACTCCGCC |
| | GACGATTGACTGAGCCAGGTTCTGTGCCTACTATCGTGA |
| | TGGTGGACGCCTACAAGCGTTACAAGGGATCCGAGACC |
| | CCGTCCCAGCGGCGCGCCACCCGCAGCGGGGCGCAGG |
| | CCAGCTC |
| GFP11-LMNA | GTCCTTCGACCCGAGCCCCGCGCCCTTTCCGGGACCCCTGC |
| | CCCGCGGGCAGCGCTGCCAACCTGCCGGCCATGCGTGACC |
| | ACATGGTCCTTCATGAGTATGTAAATGCTGCTGGGATT |
| | ACAGGATCCGAGACCCCGTCCCAGCGGCGCGCCACCCG |
| | CAGCGGGGCGCAGGCCAGCTCCACTCCGCTGTCGCCCA |
| | CCCGC |
| AFLA-LMNA | TGTCCTTCGACCCGAGCCCCGCGCCCTTTCCGGGACCCCTG |
| | CCCCGCGGGCAGCGCTGCCAACCTGCCGGCCATGCCTAGC |
| | CGCCTGGAGGAAGAACTCCGCCGACGATTGACTGAGCC |
| | AGGATCCGAGACCCCGTCCCAGCGGCGCGCCACCCGCA |
| | GCGGGGCGCAGGCCAGCTCCACTCCGCTGTCGCCCACC |
| | CGCAT |
| SpyT-LMNA | TCTGTCCTTCGACCCGAGCCCCGCGCCCTTTCCGGGACCCC |
| | TGCCCCGCGGGCAGCGCTGCCAACCTGCCGGCCATGGTGC |
| | CTACTATCGTGATGGTGGACGCCTACAAGCGTTACAAG |
| | GGATCCGAGACCCCGTCCCAGCGGCGCGCCACCCGCAG |
| | CGGGGCGCAGGCCAGCTCCACTCCGCTGTCGCCCACCC |
| | GCATC |

200 bp ultramer donor strands used for LMNA KIs. Sequences for GFP11 (green), SpyT (blue), ALFA (orange) GS linkers (black) and LMNA homology (teal) are bolded.

The above examples are provided to illustrate the invention but not to limit its scope. Other variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

1. A cell comprising:
a first fusion protein comprising a first peptide tag and a second peptide tag;
a second fusion protein comprising a first portion of a split reporter and a first affinity agent that specifically binds to the first peptide tag; and
a third fusion protein comprising a second portion of the split reporter and a second affinity agent that specifically binds to the second peptide tag,
wherein the first portion of the split reporter and the second portion of the split reporter produce a first signal when in proximity and are inactive when separate.

2. The cell of claim 1, wherein the cell expresses the first fusion protein, the second fusion protein and the third fusion protein

3. The cell of claim 1, wherein the cell expresses the first fusion protein and the second fusion protein and the third fusion protein have been introduced as proteins into the cell.

4. The cell of claim 1, wherein the peptide tags are each less than 30, 25, 15 or 10 amino acids.

5. The cell of claim 1, wherein the split reporter is a HaloTag reporter.

6. The cell of claim 1, wherein the first portion of the split reporter is an amino terminal portion of the split reporter and the first affinity agent is linked to the amino terminal side of the first portion and wherein the second portion of the split reporter is a carboxyl terminal portion of the split reporter and the second affinity agent is linked to the carboxyl terminal side of the second portion.

7. The cell of claim 1, wherein the signal is fluorescence.

8. The cell of claim 1, wherein the first peptide tag and the second peptide tag are adjacent or linked by a linker of fewer than 15 (e.g., fewer than 10, 5, 2) amino acids and are located at the amino terminus of the first fusion protein.

9. The cell of claim 1, wherein the first peptide tag and the second peptide tag are adjacent or linked by a linker of fewer than 15 (e.g., fewer than 10, 5, 2) amino acids and are located at the carboxyl terminus of the first fusion protein.

10. The cell of claim 1, wherein the first peptide tag and the second peptide tag are different and selected from the group consisting of SpyTag, SpyTag002, ALFA-tag, and GFP11 and the corresponding affinity agent is SpyCatcher if the peptide tag is SpyTag, SpyCatcher002 if the peptide tag is SpyTag002, NbALFA if the peptide tag is ALFA-tag, and GFP1-10 if the peptide tag is GFP11.

11. The cell of claim 1, wherein the first tag is GFP11 and the first affinity agent is GFP1-10, and
the second tag is SpyTag and the second affinity agent is SpyCatcher or the second tag is SpyTag002 and the second affinity agent is SpyCatcher002.

12. The cell of claim 1, wherein the first tag is ALFA-tag and the first affinity agent is NbALFA, and
the second tag is SpyTag and the second affinity agent is SpyCatcher or the second tag is SpyTag002 and the second affinity agent is SpyCatcher002.

13. The cell of claim 1, wherein the first tag is GFP11 and the first affinity agent is GFP1-10, and
the second tag is ALFA-tag and the second affinity agent is NbALFA.

14. The cell of claim 1, further comprising,
a fourth fusion protein comprising a GFP11;
a fifth fusion protein comprising a GFP1-10; and
wherein the first signal of the split reporter is distinguishable from signal from intact GFP.

15. The cell of claim 1, further comprising,
a fourth fusion protein comprising a third peptide tag and a fourth peptide tag;
a fifth fusion protein comprising a first portion of a second split reporter and a third affinity agent that specifically binds to the third peptide tag; and
a sixth fusion protein comprising a second portion of the second split reporter and a fourth affinity agent that specifically binds to the fourth peptide tag,
wherein the first portion of the second split reporter and the second portion of second the split reporter produce a signal, distinguishable from the signal of the split reporter of claim 1,
when in proximity and are inactive when separate.

16-28. (canceled)

29. A cell comprising:
a first fusion protein comprising a first peptide tag;
a second fusion protein comprising a second peptide tag;
a third fusion protein comprising a first portion of a split reporter and a first affinity agent that specifically binds to the first peptide tag; and
a fourth fusion protein comprising a second portion of the split reporter and a second affinity agent that specifically binds to the second peptide tag,
wherein the first portion of the split reporter and the second portion of the split reporter produce a signal when in proximity and are inactive when separate.

30-41. (canceled)

42. A method of measuring protein-protein interaction, the method comprising providing the cell of claim 29; and
measuring the presence or amount of the signal from the cell.

43. A cell expressing or comprising:
a first fusion protein comprising a first portion of a split reporter and a first affinity agent that specifically binds to a first peptide tag; and
a second fusion protein comprising a second portion of the split reporter and a second affinity agent that specifically binds to a second peptide tag,
wherein the first portion of the split reporter and the second portion of the split reporter produces a signal when in proximity and are inactive when separate.

44. The cell of claim 43, wherein the split reporter is a HaloTag reporter.

45. The cell of claim 43, wherein the first portion of the split reporter is an amino terminal portion of the split reporter and the first affinity agent is linked to the amino terminal side of the first portion and wherein the second portion of the split reporter is a carboxyl terminal portion of the split reporter and the second affinity agent is linked to the carboxyl terminal side of the second portion.

46-50. (canceled)
